(19) United States
(12) Patent Application Publication
(10) Pub. No.: US 2014/0011755 A1
(43) Pub. Date: Jan. 9, 2014

(54) Cardiac Glycosides for Treating Autoimmune Disease

(71) Applicants: Emily A. Stein, Redwood City, CA (US); William H. Robinson, Palo Alto, CA (US); Ricardo T. Paniagua, Dallas, TX (US)

(72) Inventors: Emily A. Stein, Redwood City, CA (US); William H. Robinson, Palo Alto, CA (US); Ricardo T. Paniagua, Dallas, TX (US)

(73) Assignees: Department of Veterans Affairs, Washington, DC (US); The Board of Trustees of the Leland Stanford Junior University, Palo Alto, CA (US)

(21) Appl. No.: 13/992,182

(22) PCT Filed: Dec. 13, 2011
(86) PCT No.: PCT/US2011/064696

§ 371 Date: Aug. 26, 2013

Related U.S. Application Data
(60) Provisional application No. 61/459,518, filed on Dec. 13, 2010.

(51) Int. Cl. A61K 31/7048; A61K 31/585

(52) U.S. Cl. CPC A61K 31/7048; A61K 31/585
USPC 514/26; 514/175

(57) ABSTRACT

The invention provides methods for treating inflammatory and autoimmune diseases by administering to the subject an effective amount of a cardiac glycoside, where the dose is effective to suppress or prevent initiation, progression, or relapses of the disease, including the progression of established disease. In some embodiments, the methods of the invention comprise administering to a subject determined to have rheumatoid arthritis or systemic lupus, or a pre-clinical disease state thereof, an effective amount of a cardiac glycoside, to suppress or prevent activity of the disease. In other embodiments, the methods of the invention comprise administering to a subject determined to have insufficient endogenous cardiac glycosides, an effective amount as a supplement to counteract the effects of cardiac glycoside deficiency.

### FEDERALLY SPONSORED RESEARCH AND DEVELOPMENT

This invention was made with Government support under grant no. N01-HV-28183 awarded by the National Institutes of Health. The Government has certain rights in this invention

### BACKGROUND OF THE INVENTION

There is a long-standing interest in manipulating cells of the immune system to achieve control of autoimmune disease. The pathology of autoimmune diseases is mediated by aberrant responses of multiple immune and other cell types. In addition to general immunosuppression, e.g. through the use of agents such as hydrocortisone, many therapies are now being brought to the clinic that provide for a more selective modification of the immune system and cell responses to inflammation, such as blockade of cytokines TNF, IL-1, IL-6, and IL-15; reduction of B cell populations, T cell populations; inhibiting T cell activation; or altering interactions of adhesion or signaling molecules prominent in inflammation.

B cells are responsible for producing autoantibodies, which contribute to the pathogenesis of diseases previously thought to have a largely T cell pathology, for example, rheumatoid factors (RF) and other RA-associated autoantibodies such as anti-cyclic citrullinated peptide (CCP) antibodies in rheumatoid arthritis; and anti-nuclear antibodies in systemic lupus erythematosus (SLE); and anti-acetylcholine receptor (AChR) antibodies in myasthenia gravis; and anti-insulin, anti-glutamic acid decarboxylase (GAD) and IA2 antibodies in type 1 diabetes; and anti-myelin antibodies in multiple sclerosis. B cells also act as highly efficient antigen-presenting cells (APC) to T cells and thus may play an important role in T cell activation.

Cytokines play roles in the pathogenesis of rheumatoid arthritis, systemic lupus erythematous, myasthenia gravis, and other autoimmune diseases. Cytokines include chemokines, interleukins, lymphokines, growth factors, angiogenesis factors, and other secreted and cell surface molecules that transmit signals to other cells. Cytokines include members of the tumor necrosis factor (TNF) family. Blockade of cytokines with biological agents such as monoclonal antibodies and soluble receptors targeting TNF have already provided therapeutic benefit in autoimmune diseases including RA, psoriasis and Crohn's disease.

Cardiac glycosides are a structurally diverse and large family of cholesterol-based naturally synthesized molecules that can be separated into two classes based on a 5-carbon ring (cardenolide) or a 6-carbon ring (bufadienolide) attached to position 17 of the steroid core. Cardiac glycosides are also referred to as cardiotonic steroids, cardiotonic genins, and digitalis glycosides. The pharmacokinetic/pharmacodynamic properties vary considerably among cardiac glycosides and are conferred by the sugar moiety attached to position 3 of the steroid core. The biological functions of cardiac glycosides are not well understood, but much is known about the role of cardiac glycosides as natural ligands for the Na⁺/K⁺-ATPase. Cardiac glycosides specifically bind and inhibit the catalytic α-subunit of the Na⁺/K⁺-ATPase, causing an increased intracellular Na⁺ concentration which leads to an overall increase in intracellular calcium ions. Emerging data suggests a physical role for cardiac glycosides inducing conformational changes in the Na⁺/K⁺-ATPase that trigger ion pump-independent activation of the Na⁺/K⁺-ATPase signalosome.

Novel uses of cardiac glycosides are provided herein.

### SUMMARY OF THE INVENTION

Compositions and methods are provided for the treatment of inflammatory disease in an individual, e.g. including B cell-associated diseases, e.g. rheumatoid arthritis, systemic lupus erythematosus, and myasthenia gravis, by administering to the individual a dose of a cardiac glycoside by a route and dosing regimen that is effective in increasing expression of at least one anti-inflammatory cytokine by an endogenous immune cell, which cytokines include without limitation IL-10 and TGF-β. Increase in expression may be at least about 2-fold, at least about 3-fold, at least about 5-fold, or more, relative to an untreated control. Immune cells of interest include lymphocytes, e.g. B lymphocytes, as well as monocytes and macrophages.

The data provided herein demonstrate that cardiac glycosides induce activity of regulatory B cells, which secrete IL-10 and TGFβ, and down-regulate IL-6 expression. Cardiac glycosides of interest include those of the cardenolide and bufadienolide classes, and include naturally occurring and synthetic compounds. In addition to plant-derived compounds, such as digitoxin, ouabain, oleandrin, etc., of interest are cardiac glycosides naturally present in mammals, e.g. in humans, such as ouabain, digoxin, proscillaridin, telocinobufagen, and the like.

The methods of the invention further comprise administering to the host an effective amount of a cardiac glycoside, where the dose is effective to suppress or prevent initiation, progression, or relapses of disease, including the progression of established disease. In some embodiments, the methods of the invention comprise administering to a subject having a pre-existing inflammatory disease condition, an effective amount of a cardiac glycoside, to suppress or prevent relapses of the disease. In some embodiments of the invention, the cardiac glycoside is administered orally. Sublingual administration is of particular interest. In some embodiments, a patient is diagnosed to have an autoimmune disease, e.g. rheumatoid arthritis, systemic lupus erythematosus, or myasthenia gravis by a suitable diagnostic method. Patients may be further selected on the basis of endogenous cardiac glycoside levels, where individuals with low levels relative to a reference normal sample may find the methods of the present invention particularly useful. Dosing may be advantageously performed at a frequency less than daily, e.g. every two days, every three days, etc.

In some embodiments the cardiac glycoside may be combined with a second anti-inflammatory agent, where the combination provides for a synergistic effect. The combination may allow for use of a reduced dose of one or both agents. In some embodiments the second agent inhibits TNFR signaling. In other embodiments the second agent is a disease modifying anti-rheumatic agent other than a TNFR inhibitor.

Methods are also provided for the design and dose optimization of cardiac glycosides for use in the treatment of inflammatory diseases. Such methods may include providing candidate compounds or doses, including without limitation analogs and derivative of known cardiac glycosides, contacting a cell population comprising B cells with the candidate compound or dose, and determining the effect on development of regulatory B cells, including the detection of cytokines IL-10, TGFβ and IL-6. The population of B cells may be provided in an in vitro culture model, or in an in vivo model, e.g. various animal models for inflammatory diseases. Agents and dosages that increase the number or effectiveness of regulatory B cells are of interest.

Pharmaceutical compositions are provided, which comprise a dose of a cardiac glycoside effective to stimulate the generation of regulatory B cells, including without limitation oral formulations, particularly sublingual formulations, in a unit dose appropriate for the methods of the invention.

### BRIEF DESCRIPTION OF THE DRAWINGS

FIG. 1. Structural features of cardiac glycosides. (A, B) Structure of cardiac glycosides, illustrating the steroid core (aglycone portion), the glycoside (glycone or sugar portion) and the R group (5 or 6 member carbon ring that defines the bufadienolide and cardenolide classes of cardiac glycosides, respectively). (C) Tertiary structure of the R group of cardiac glycosides, (D) which forms a “U”-shaped molecular structure.

FIG. 2. Structures of the cardenolide and bufadienolide classes of cardiac glycosides. There are two major classes of cardiac glycosides based upon whether a 5-carbon (cardenolide) or 6-carbon (bufadienolide) ring structure is present at the R group (FIG. 1). Members of both cardenolide and bufadienolide classes have been isolated from plants and humans.

FIG. 3. Examples of cardiac glycosides and substitutions on the aglycone core. Cardiac glycosides were first discovered in plants and many of the names are derived from the plant source. Digitalis purpurea, Digitalis lanata, Strophanthus grtus, Strophanthus kombe are major plant sources of cardiac glycosides. The term “genin” at the end of a cardiac glycoside name refers only to the aglycone moiety. Many genins have hydroxyls groups at the 12- and 16-positions. These additional hydroxyl groups greatly influence the pharmacokinetic profile of the cardiac glycoside.

FIG. 4. Cardiac glycosides prevent and treat the collagen-induced arthritis (CIA) mouse model for rheumatoid arthritis. (A-D), Cardiac glycosides including digitoxin 0.3 mg/kg (A, B), 0.03 mg/kg ouabain (C, D) or proscillaridin 0.25 mg/kg (C, D) were administered daily by intra-peritoneal injection starting the day prior to induction of CIA. CIA was induced by intradermal immunization with bovine type II collagen emulsified in complete Freund's adjuvant, followed at day 21 by boosting with type II collagen emulsified in incomplete Freund's adjuvant. Mean Visual Arthritis Score (A, C) or Mean Paw Thickness (B, D) are graphed on the Y axis, and Days Post-Immunization on the X axis. Statistical comparisons were performed using the Mann-Whitney test and asterisks represent P-values less than 0.01. Error bars represent the standard error of the mean (SEM). Digitoxin, ouabain and proscillaridin all prevented the development of CIA. (E, F) The cardiac glycosides digitoxin 0.3 mg/kg and proscillaridin 0.25 mg/kg were administered daily by intra-peritoneal injection to mice with established arthritis, treatment was initiated in mice when they reached a Mean Visual Score of 2. Mann-Whitney test demonstrate statistically significant reductions in both the Mean Visual Score and Mean Paw Thickness in the cardiac glycoside treated mice.

FIG. 5. Multiple cardiac glycosides effectively reduced the severity of the collagen arthritis mouse model of rheumatoid arthritis. (A) Representative prevention study demonstrating that mice receiving 0.3 mg/kg digitoxin (squares), 0.03 mg/kg ouabain (triangles), 0.25 mg/kg proscillaridin (mark), 0.35 mg/kg digoxin (“x”) and 0.15 mg/kg bufalin (dash) have significantly reduced average clinical arthritis scores compared to vehicle mice. (B) Average paw thicknesses of recipient mice were determined using calipers to measure hind paw thickness. 15 mice per group were enrolled and data was analyzed for statistical significance using the Mann-Whitney test with one asterisk indicating p<0.05 and two asterisks indicating p<0.01.

FIG. 6. Sublingual dosing every 3 days is effective in preventing the collagen arthritis mouse model of rheumatoid arthritis. (A, B) Data from a prevention study where mice were dosed once prior to boost with 0.9 mg/kg digitoxin (squares) or 0.6 mg/kg ouabain (diamonds) by intraperitoneal injections on days −3, 0, 3, 6, 9 and 12 after boost. (A) The average clinical scores of mice in each group. (C) The average paw thickness measured in each group. (B, D) Data from a prevention study where mice were dosed once prior to boost with 0.9 mg/kg digitoxin (squares) or 0.6 mg/kg ouabain (diamonds) by sublingual administration on days −3, 0, 3, 6, 9 and 12, after boost. (B) The average clinical scores of mice in each group. (D) The average paw thickness measured in each group. (E-G) Data from a dose titration experiment where mice were dosed daily starting on day −1 from boost with 0.1 or 0.4 mg/kg digitoxin, (E), 0.05 or 0.2 mg/kg ouabain (F), and 0.1 or 0.4 mg/kg proscillaridin (G) by intraperitoneal administration. Data was analyzed for statistical significance using Mann-Whitney test with one asterisk indicating P<0.05.

FIG. 7. Cardiac glycosides reduce joint pathology in the CIA mouse model for rheumatoid arthritis. Blinded scoring was performed on histologic sections of joints harvested from mice at the end of a CIA prevention study (A-C). Joints derived from three representative mice belonging to each prevention group (A-C) were decalcified, fixed and stained with tolidine blue, followed by scoring by an examiner blinded to treatment status for the severity of synovitis (inflammation), pannus (growth of the synovial lining), and erosions of bone. (D-G) Joints derived from three representative mice belonging to each prevention group were decalcified, fixed, and stained with hematoxylin and eosin. Cross-sectioned ankle joints from vehicle (D), digitoxin (E), ouabain (F) and proscillaridin (G) treated mice are presented. Error bars represent SEM, and asterisks indicate P-values less than 0.05 by Mann-Whitney test. FIGS. 5D and E are representative toludine blue-stained sections of joints derived from vehicle control (D) and digitoxin (E) treated mice with CIA.

FIG. 8. Cardiac glycosides demonstrate a class effect in the ability to inhibit TNFR-mediated NFkB activity. (A) HeLa cells were co-transfected with an NFκB reporter (pNFκB) or control vector (pTAL), and following pre-treatment for 6 hours with 0-500 nM of various cardiac glycosides (VC, vehicle control; DT, digitoxin; DO, digoxin; OA, ouabain; PR, proscillaridin A; BF, bufalin) cells were stimulated with 50 ng/mL TNF. Values represent mean light units produced. Error bars represent SEM, and statistical significance was determined by unpaired T-test. Single and double asterisks represent P-values of 0.01 or 0.001, respectively. All cardiac glycosides tested reduced TNF-mediated NFκB activation in a dose-dependent fashion. (B) Western blot of lysate taken from CIA B cells pre-treated with vehicle or 33 nM digitoxin for 1 h prior to stimulation with 50 ng/mL CD40L. Samples were harvested at designated time points and were fractionated into nuclear and cytoplasmic lysates (Pierce). Lysates were quantified using BCA (Pierce), and were equally loaded according to total protein. Membranes were probed for levels of nuclear phospho-p65 (ReIA), phospho-ReIB, and histone (histone served as loading control and fractionation method control) and cytoplasmic degradation of IκBα (beta-tubulin served as loading control and fractionation method control). Digitoxin significantly reduced the amount of nuclear phospho-p65 minutes following CD40L stimulation.

FIG. 9. Serum cytokine levels are similar between vehicle and cardiac glycoside treatment groups. Serum from 3 mice per treatment group (vehicle, digitoxin, ouabain, proscillaridin, digoxin and bufalin) was assessed in triplicate for TNF, IL-6 and IL-1beta levels using ELISA. The unpaired student t-test was used to assess significance.

FIG. 10. Cardiac glycosides reduce immunoglobulin levels in CIA mice. ELISAs for anti-type II collagen antibodies were performed on sera derived from 6 representative mice of each prevention group (A, B). Isotype specific antibodies for IgG1 and IgG2a (A) and IgM (B) were utilized. Treatment with digitoxin, ouabain and proscillaridin all reduced levels of both IgG1 and IgM relative to vehicle-treated control mice. Statistical analysis was performed using ANOVA and asterisks indicate points at which the P-values were less than 0.001.

FIG. 11. Cardiac glycosides inhibit cellular responses mediated by the TNFR family members CD40, TNFR and BAFF, but do not modulate cellular responses mediated through the T or B cell receptors. (A) Digitoxin inhibits the proliferation of unstimulated or collagen II-stimulated splenocytes derived from mice with CIA. (B) Cardiac glycosides, including digitoxin, digoxin and ouabain, did not alter proliferation of T cells in response to stimulation through the T cell receptor (a non-TNFR family member). (C) Pre-treatment with 3.3 or 33 nM of the cardiac glycosides digitoxin, ouabain and proscillaridin inhibits TNF or CD40L-mediated activation of macrophage through TNFR and CD40 (both TNFR family members), respectively. (D) Pre-treatment of B cells with 33 nM digitoxin inhibited BAFF-mediated stimulation (0.5 or 5 μg/ml) through BAFFR (a TNFR family member). (E) Pre-treatment of B cells with digitoxin prevented anti-CD40 mediated B cell proliferation through CD40 (a TNFR family member). (F) Pre-treatment of B cells with digitoxin inhibited CD40L-mediated proliferation, but did not inhibit B cell receptor (membrane Ig, a non-TNFR family member) mediated proliferation.

FIG. 12. Cardiac glycosides inhibit signaling mediated by multiple TNFR family receptors. The results of studies with multiple cell lines and primary cells treated with cardiac glycosides and stimulated with ligands for TNFR family receptors, or for non-TNFR family receptors, are summarized. TNFR1/R2, NGFR, CD40, and BAFFR/TACI/BCMA are all members of the TNFR family. The B cell receptor (BCR), T cell receptor (TCR), CD28, protein kinase C pathway (PKC) and lectin-binding pathway are all non-TNFR family receptors/pathways. Cardiac glycosides inhibited signaling mediated by all TNFR family receptors tested (boxed), but did not inhibit signaling through the non-TNFR family receptors/pathways tested.

FIG. 13. Endogenous cardiac glycoside levels are reduced in human rheumatoid arthritis patient sera compared to healthy individuals. (A) Endogenous cardiac glycoside levels were determined in sera from four CCP− and nine CCP+ rheumatoid arthritis patients and were compared to sera from 14 healthy individuals, using an ELISA. Results have been validated in the ARAMIS cohort with similar trends. (B) Endogenous ouabain levels are significantly lower in CCP+ RA patient sera. 0.9 mL of human plasma from patients with rheumatoid arthritis and healthy controls were spiked with cymarin at 5 ng/sample to serve as an internal standard. Samples were then precipitated with 0.4 mL of 0.1% formic acid in acetonitrile followed by centrifugation and evaporation. Samples were reconstituted in 0.1 mL acetonitrile/0.1% formic acid at 1:9 molar ratio. Endogenous cardiac glycoside isolation was performed with the HP1100 HPLC system (Agilent Technologies, Santa Clara, Calif.) and MS/MS was performed using a Quattro Premier triple quadropole mass spectrometer equipped with an electrospray probe (Waters, Milford, Mass.). The Mann-Whitney U-test was used to assess significance.

FIG. 14. Cardiac glycosides inhibit signaling mediated by members of the TNFR family, and result in the inhibition of NFκB activity in response to the stimulation from TNFR ligands. The TNFR family is a major receptor family that responds to specific ligands to activate the downstream transcription factor NFκB. The ability of cardiac glycosides to inhibit cellular activation by multiple TNFR family members results in immune modulation that could provide benefit in inflammatory diseases.

FIG. 15. Cardiac glycosides stimulate STAT3 phosphorylation in B cells, macrophage and splenocytes from CIA mice. (A) Splenic B cells from CIA mice were incubated in the presence of vehicle or 33 nM digitoxin for 30 minutes prior to. fixation and staining with antibodies against phospho-STAT3 (Y705) (Cell Signaling Technologies). Flow cytometry was performed using the FACSAria II (Bectin Dickenson). (B) Splenic B cells were incubated in the presence of 33 nM digitoxin for 0, 5, 15, 30, 45, and 60 minutes and were harvested using cell lysis buffer (Pierce). Lysates were quantified for total protein using the Nanodrop, and verification was performed by BCA (Pierce). Lysates were loaded onto SDS-PAGE and transferred to PVDF (Millipore). Membranes were blotted for phospho-STAT3 (Y705) and beta-actin.

FIG. 16. Cardiac glycosides decrease disease severity in experimental lupus. SLE-like disease progression in MRL-Ipr female mice (Jackson Laboratories) was evaluated. Briefly, 10 mice per treatment arm were followed for the development of SLE-like disease over the course of 16 weeks. At week 8, dosing was initiated with vehicle or 0.6 mg/kg digitoxin every-4-days by sublingual administration. Animals were scored on a weekly basis for proteinuria and survived until 23 weeks of age. Data represent the average proteinuria concentration in urine collected from each mouse using Uristix (Bayer Corp.). Error bars represent SEM. The Mann-Whitney test was used to determine significance between vehicle and digitoxin groups. Asterisks represent significant P values <0.01.

FIG. 17. Cardiac glycosides decrease kidney pathology in experimental lupus. At endpoint, 23 week old female MRL-Ipr mice were sacrificed and kidneys were fixed, embedded in paraffin, sectioned longitudinally and stained for glomeruli with periodic acid-Schiff stain (Histotec). (A) Depiction of a section taken from a representative vehicle treated kidney. (B) Depiction of a section taken from a representative digitoxin treated kidney.

FIG. 18. Cardiac glycosides inhibit autoantibody production. Endpoint serum titers were taken for each mouse and the concentration of anti-double-stranded DNA (ANA) antibodies were assessed (A). Additional tests were performed on 2×10⁵ vehicle-treated MRL-Ipr splenic B cells to determine the ability of digitoxin (33 nM) to inhibit ANA production in vitro at 72 hours (B). Digitoxin-specific blockade of autoantibody production was relieved when B cells were pre-treated with 0.8 nM mifepristone, the established IC₅₀ for the glucocorticoid receptor. ANA antibodies were measured using ELISA (Alpha Diagnostic International). Error bars represent the SEM. The student t-test was used to determine significance. Asterisks represent significant P values.

FIG. 19. Cardiac glycoside induces IL-10 cytokine secretion from B cells in a mechanism dependent on steroid receptors, Jak activity, and STAT3 activity, but not BCR-ABL or Src-like Family Kinase activity. MACS-purified B cells from vehicle or digitoxin-treated 23 week-old female MRL-Ipr mice were harvested from the spleen. 2×10⁵ B cells were incubated either media, mifespristone (0.8 nM), Jak inhibitor I (15 nM), STAT3 inhibitor VI (500 μM) or dasatinib (1.1 nM) for 60 minutes, washed and incubated for 48 hr at 37° C. Cell supernatant was collected and ELISAs were used to quantify the amount of secreted IL-10 cytokine in each condition. Data represent the average of triplicate conditions and error bars represent the SEM. Statistical significance was performed using the student t-test.

FIG. 20. Cardiac glycoside induces STAT3-dependent IL-10 gene expression in B cells. (A and B) 1×10⁶ splenic B cells, dendritic cells, or monocyte/macrophage were purified from CIA mice (MACS, magnetic cell separation) and were treated with vehicle or digitoxin (33 nM) for 30 minutes prior to mRNA harvest. IL-10 (FIG. 20 A) and TGFβ1 (FIG. 20 B) levels were quantified. Samples were normalized against Ywhaz1 and Hprt1 housekeeping genes. Data represents the average fold expression levels in digitoxin-treated cells over vehicle-treated expression levels. Samples were examined in triplicates and the error bars represent the SEM. (C) 1×10⁶ B cells from CIA mice were treated with media, digitoxin (33 nM), STAT3 inhibitor VI (500 μM) or digitoxin and STAT3 inhibitor VI. After 30 minutes, cells were washed, lysed and mRNA was isolated with Trizol (Invitrogen). RT-PCR was performed on mRNA and samples were normalized against housekeeping genes, Ywhaz1 and Hprt1. Data represent the average fold change in IL-10 gene expression compared to media alone. Error bars represent SEM of triplicates. (D) 2×10⁵ B cells from CIA mice were treated with vehicle or digitoxin (33 nM) and incubated for 48 hr. IL-10 protein levels were measured by ELISA (eBiosciences). Data represent averages of triplicates. Error bars represent SEM.

FIG. 21. Therapeutic levels of cardiac glycoside induce IL-10, TGFβ and inhibit IL-6 expression in B cells. (A) 1×10⁶ B cells from CIA mice were treated with media or digitoxin (33 nM and 100 nM) and incubated for 45 minutes prior to mRNA isolation. RT-PCR was performed and TGFβ1, IL-10 and IL-6 mRNA levels were quantified. Sample cDNA levels were normalized against housekeeping genes Ywhaz1 and Hprt1. Data represent the average fold change in IL-10 gene expression compared to media alone. Error bars represent SEM of triplicates. (B) 1×10⁶ B cells from CIA mice were treated with media or digitoxin (33 nM) and gene expression of IL-10, IL-6, TGFβ1, SOCS3, IκBα, LPS binding protein (LBP) were analyzed. Data represent fold changes in mRNA levels of digitoxin-treated cells compared to vehicle.

FIG. 22. Cardiac glycoside-specific effects on B cells are divergent from a classical anti-inflammatory effects of corticosteroids. 1×10⁶ B cells from CIA mice were treated with media or digitoxin (33 nM) or dexamethasone (2 mM) and incubated for 45 minutes prior to mRNA isolation. RT-PCR was performed and TGFβ1 and IL-10 levels were quantified and samples were normalized against Ywhaz1 and Hprt1. Data represent the average fold gene induction of triplicates and error bars represent SEM.

FIG. 23. Cardiac glycoside treatment induced IL-10 and TGFβ expression, and down-regulation of IL-6 expression, in B cells is dependent on STAT3 activity. (A) Representative data from CIA B cells were treated with vehicle, digitoxin (33 nM), IL-6 (2 ng/mL), IL-10 (10 ng/mL) or STAT3 inhibitor VI (500 μM) and digitoxin (33 nM). Cell lysates were harvested at 48 hr and IL-6 and IL-10 were quantified. (B) TGFβ levels were also assessed. Data represents the average absorbance of triplicates relative to vehicle. Error bars represent SEM. IL-6 levels were not determined for IL-6-treated samples and IL-10 levels were not determined for IL-10-treated samples.

FIG. 24. Cardiac glycoside-treated B cells down-regulates Th17 cell differentiation. (A) Representative data from T cell differentiation studies whereby 5×10⁵ MACS (magnetic cell separation)-purified CIA B cells were treated with vehicle or digitoxin (33 nM) for 30 minutes, washed and then added to MACS-purified naïve CD4⁺ T cells in the presence of Th17 T cell differentiation media (complete RPMI supplemented with anti-CD3 and anti-CD28 antibodies (4 μg/mL), IL-6 (80 ng/mL) and TGFβ (1 ng/mL)) and incubated for 72 hr. At endpoint, IL-17 cytokine levels were quantified by ELISA (R&D Systems). (B) IL-10 cytokine production was also assessed in each sample by ELISA (eBiosciences). Data represent the average of triplicates and error bars represent the SEM. Statistical significance was determined by the students t-test.

FIG. 25. Cardiac glycoside-treated B cells induce regulatory T cells in vitro and in vivo. (A) Representative data from mixed lymphocyte studies. Briefly, 1×10⁵ purified CIA B cells were treated with vehicle, digitoxin (33 nM) or STAT3 inhibitor VI (500 μM)+digitoxin (33 nM) for 1 hr. B cells were washed, replated in complete RPMI supplemented with BAFF (1 ng/mL), anti-CD3 antibody (4 μg/mL) and were added to 5×10⁵ naïve CD4⁺ T cells purified by MACS. Cells were incubated for 72 hr prior to harvest. IL-6, IL-10, TGFβ and IL-17 cytokine levels were measured by ELISA (R&D Systems, Peprotech, and eBiosciences). Data represent the average absorbance (A₄₅₀) of triplicates and error bars represent the SEM. (B) Representative data from splenocytes isolated from DBA1/J naïve, vehicle or digitoxin (0.3 mg/kg) treated CIA mice. Cells were fixed in 4% paraformaldehyde for 15 minutes, washed twice with PBS and blocked with FACS buffer. Cells were stained for regulatory T cells using anti-TCRβ, anti-CD4, anti-CD25 and anti-FoxP3 antibodies (Cell Signaling Technologies, R&D Systems, AbCam). FACS was performed on the FACS Aria (Becton-Dickenson). Data represent the average population in 500,000 cells from 4 mice per treatment group. Error bars represent the SEM.

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended clairheumatoid arthritis, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### DEFINITIONS

“Cardiac glycosides” are an important class of naturally occurring or synthetic drugs. A select set of cardiac glycosides are used in the treatment of congestive heart failure and for treatment of atrial fibrillation and flutter, and include a number of clinically used compounds. Cardiac glycosides comprise molecules synthesized by plants, molecules synthesized by humans and other mammals, and synthetic derivatives of these molecules. Cardiotonic steroids (CS) are the cardiac glycosides produced by humans and other mammals.

The pharmacology of cardiac glycosides is known in the art, for example see Prassas et al. (2008) Nat Rev Drug Discov. 7(11):926-35; Nesher et al. (2007) Life Sci. 80(23):2093-107; and Gupta (2000) Prog Drug Res. 55:235-82, each herein specifically incorporated for reference).

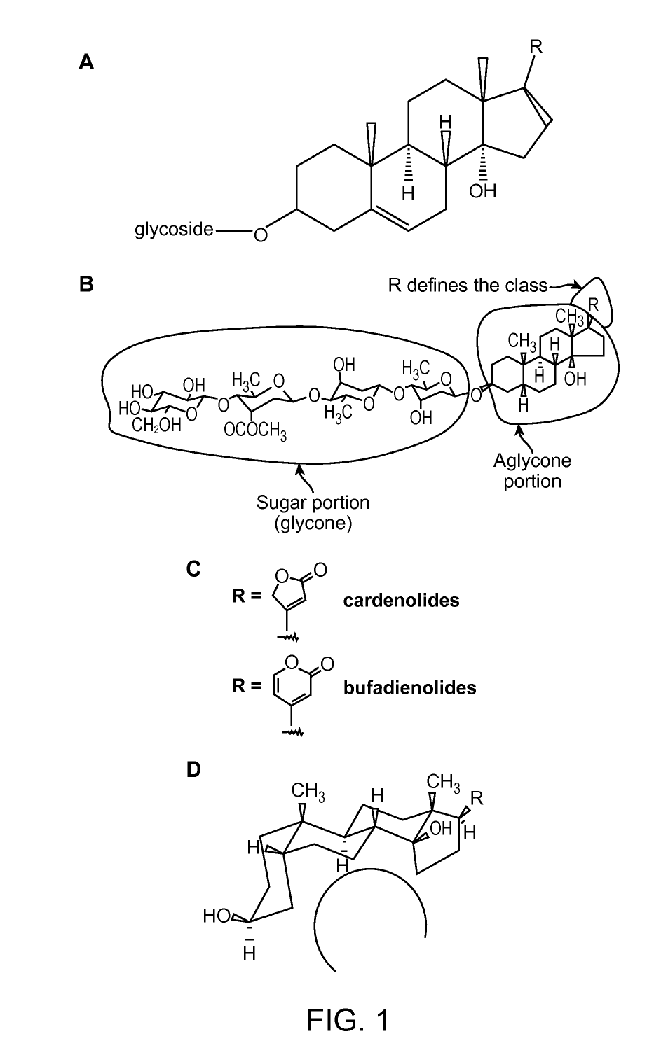

Cardiac glycosides are composed of two structural features: the sugar (glycoside) and the non-sugar (aglycone, steroid) moieties (FIG. 1A). The steroid nucleus has a unique set of fused ring system that makes the aglycone moiety structurally distinct from the other more common steroid ring systems. Rings A/B and C/D are cis fused while rings B/C are trans fused. Such ring fusion gives the aglycone nucleus of cardiac glycosides the characteristic ‘U’ shape. For example, the structure may be as shown in FIG. 1A, where a glycoside is linked to a steroid core; and the R group is a 5-membered ring or a 6-membered ring (bufadienolide).

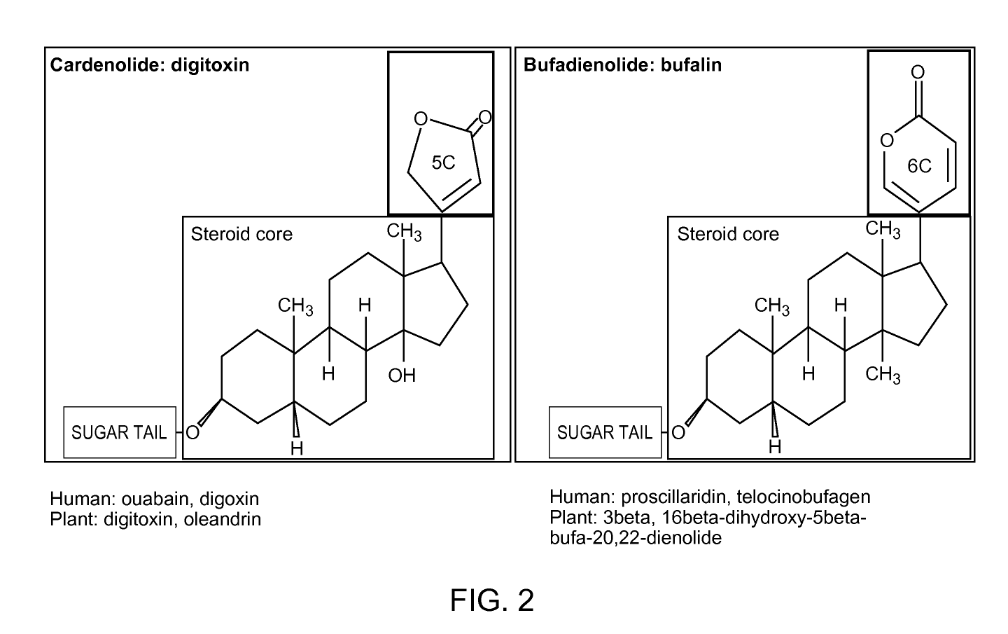

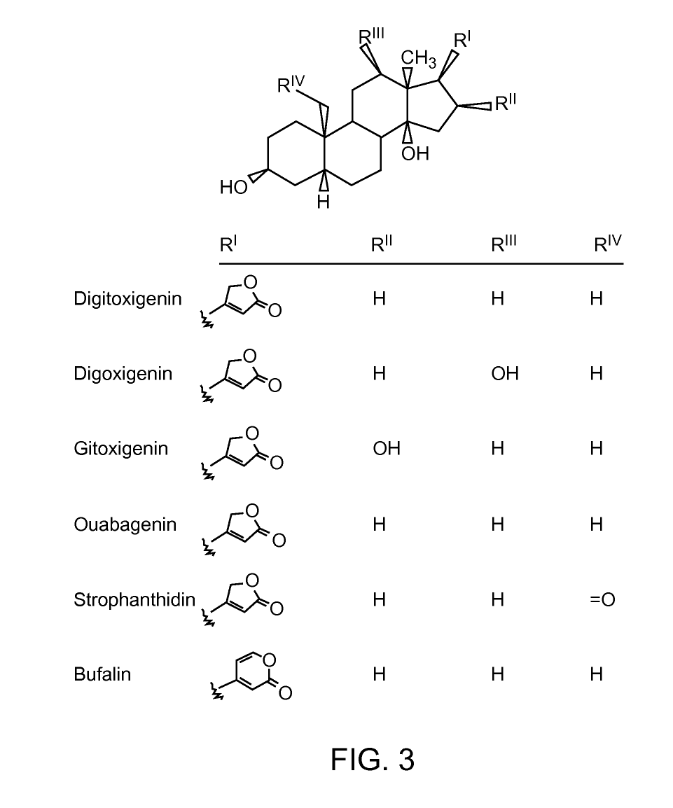

The R group at the 17-position defines the class of cardiac glycoside. Two classes have been observed in nature—the cardenolides and the bufadienolides. The cardenolides have an unsaturated butyrolactone ring while the bufadienolides have an a-pyrone ring (FIG. 1B). The cardiac glycosides were originally found in plants, from which the names have been derived. More recently, cardiac glycosides have been identified to be produced by the adrenal glands, hypothalamus, and other endocrine and non-endocrine organs (for example, the heart under conditions of anoxia). The term ‘genin’ at the end refers to only the aglycone portion (without the sugar; FIG. 1A steroid core without the R or glycoside attachments). Thus the word digitoxin refers to a agent consisting of digitoxigenin (aglycone) and sugar moieties (three). The steroid structures of some cardiac glycosides of interest are presented in FIG. 2.

The steroid nucleus has hydroxyls at 3- and 14-positions of which the sugar attachment uses the 3-OH group (FIG. 2). 14-OH is normally unsubstituted. Many genins have OH groups at 12- and 16-positions. These additional hydroxyl groups influence the partitioning of the cardiac glycosides into the aqueous media and greatly affect the duration of action.

The lactone moiety at C-17 position is an important structural feature. The size and degree of unsaturation varies with the source of the glycoside.

One to 4 sugars are found to be present in most cardiac glycosides attached to the 3b-OH group. The sugars most commonly found include L-rhamnose, D-glucose, D-digitoxose, D-digitalose, D-digginose, D-sarmentose, L-vallarose, and D-fructose. These sugars predominantly exist in the cardiac glycosides in the b-conformation. The presence of acetyl group on the sugar affects the lipophilic character and the kinetic profile of the entire glycoside. The sugar moiety is important for the partitioning and kinetic profile of action.

Commercially available cardiac steroids differ markedly in their degree of absorption, half-life, and the time to maximal effect.

| Agent | GI absorption | Onset (m) | Peak (h) | Half-life |
|---|---|---|---|---|
| Ouabain | | 5-10 | 0.5-2 | 21 h |
| Deslanoside | | 10-30 | 1-2 | 33 h |
| Digoxin | 55-75% | 15-30 | 1.5-5 | 36 h |
| Digitoxin | 90-100% | 25-120 | 4-12 | 4-6 days |

These differences may be due to the polarity differences caused by the number of sugars at C-3 and the presence of additional hydroxyls on the cardenolide. Although two cardiac glycosides may differ by only one sugar residue their partition co-efficients may be significantly different resulting in different pharmacokinetic profiles. For example, lanatoside C and digoxin differ only by a glucose residue and yet the partition co-efficient measured in CHCI₃/16% aqueous methanol (MeOH) are 16.2 and 81.5, respectively.

| | Partition |
|---|---|
| Glycoside | Coefficient |
| Lanatoside C (glucose-3-acetyldigitoxose-digitoxose₂- | 16.2 |
| digoxigenin) | |
| Digoxin (digitoxose₃-digoxigenin) | 81.5 |
| Digitoxin (digitoxose₃-digitoxigenin) | 96.5 |
| Acetyldigoxin (3-acetyldigitoxose-digitoxose₂-digoxigenin) | 98.0 |
| G-Strophanthin (rhamnose-ouabagein) | very low |

In general, cardiac glycosides with more lipophilic character are absorbed faster and exhibit longer duration of action as a result of slower urinary excretion rate. Lipophilicity is markedly influenced by the number of sugar residues and the number of hydroxyl groups on the aglycone part of the glycoside.

“Activity” of cardiac glycoside shall mean any biological or binding function performed by that compound.

“Comparable cell” shall mean a cell whose type is identical to that of another cell to which it is compared. Examples of comparable cells are cells from the same cell line.

“Inhibiting” the onset of a disorder shall mean either lessening the likelihood of the disorder's onset, or preventing the onset of the disorder entirely. In the preferred embodiment, inhibiting the onset of a disorder means preventing its onset entirely. As used herein, onset may refer to a relapse in a patient that has ongoing relapsing remitting disease. The methods of the invention are specifically applied to patients that have been diagnosed with an autoimmune disease. Treatment is aimed at the treatment or prevention of relapses, which are an exacerbation of a pre-existing condition. Treatment can also prevent progression of disease symptoms. For the purposes of the present invention, diagnosis of a pre-clinical state is of interest, where patients are selected for treatment by the methods of the present invention based on the presence of markers indicative of a high level of predisposition to disease. For example see co-pending U.S. patent application Ser. No. 12/214,670, herein specifically incorporated by reference.

“NF-κB” is a heterodimeric transcription factor that controls the expression of genes involved in cell adhesion, cell cycle progression, inhibition of apoptosis and pro-inflammatory responses. The classical NF-κB pathway is initiated by TNF binding to the receptor TNFR1, followed by association of the scaffolding protein TRADD, which promotes the recruitment TABS and TRAFs to the TNF-TNFR1/TRADD complex. Activation of TAK1 and downstream kinases leads to the activation of the Iκκ kinase complex. Active Iκκs phosphorylate the NF-κB inhibitor complex, IκB, leading to the ubiquitination and degradation of the IκB by the 26S proteosome. Perturbation of NFκB-dependent gene expression has been demonstrated in cases of altered IκB or IκBα levels (Barken et al, (2005) Science 308:52a. and Beg et al, (1995) Genes and Dev 9:2736-2746). Free cytoplasmic NF-κB is processed and the active form translocates to the nucleus. NF-κB transcriptional activity culminates in the expression of cytokines such as IL-1, TNF, IL-6, IL-12, IL-18, which are present in high concentrations in the rheumatic joint. Interestingly, in some RA and other autoimmune disease patients NF-κB is constitutively activated. NF-κB inhibitors and NF-κB-deficient dendritic cells suppress active disease in animal models of RA. NF-κB activity also stimulates the proliferation of FLS and promotes the highly motile, matrix metalloprotease-secreting phenotypes characteristic of a highly invasive pannus. During TNFR1 signaling, NF-κB is one of the principal signaling pathways, and the activation of IKK requires some of the key adaptor proteins such as TRADD, TRAF2, and RIP.

“Signal transducer and activator of transcription” (STAT) are transcription factors that are maintained in an inactive state near the cytoplasmic membrane. STATs are activated when phosphorylated by membrane-proximal kinases such as JAKs and Src, which are activated upon cytokine engagement with the respective receptor. STAT phosphorylation promotes nuclear translocation to whereby STATS regulate the transcription of genes containing GAS sequences in the promoter region. STATs have also been demonstrated to form heterodimers with p50 and p65 of NFκB and other transcription factors. STAT-containing heterodimers have also been demonstrated to bind to and regulate the transcription of genes that do not contain GAS sequences in the promoter region.

“STAT3” The transcription factor, signal transducer and activator of transcription 3 (STAT3), acts downstream of cytokine and growth factor receptors such as EGFR, IL6R, IL10R and others to play a critical role in inflammation, development, cell growth, and homeostasis and has been demonstrated to play a role in certain cancers and autoimmune diseases (Hirano et al, 2000; Levy and Lee, 2002; Murray, 2006). STAT3 activity is regulated by phosphorylation at residues S⁷²⁷ and Y⁷⁰⁵ by the JAK family of kinases, Src and other kinases. Numerous examples demonstrate the ability for STAT3 phosphorylation to be uncoupled, with cytokine-stimulated phosphorylation occurring on one residue without observed phosphorylation on the other residue (Ceresa and Pessin, 1996; Gotoh et al, 1996; Kovarik et al, 1998; Kuroki and O'Flaherty, 1999; Lim and Cao, 1999; Ng and Cantrell, 1997). Phosphorylation of STAT3 at Y⁷⁰⁵ leads to its dimerization, nuclear translocation, DNA binding and gene transcription. STAT3 regulates the expression of genes that promote survival (bcl-xl, survivin), proliferation (cyclin D1), invasion (matrix metalloproteinase-2) and angiogenesis (VEGF). Additionally, STAT3 can play anti-inflammatory roles and is dependent on the cellular context (Cui et al, (2011) Immunity 35:792-805; Horiguchi et al, (2008) Gastroent 134:1148-1158). STAT3 phosphorylation at S⁷²⁷ has been implicated in modulating oxidative stress response and is associated with cellular transformation.

“Cytokine” is any number of small protein, peptides and glycoprotein signals that are secreted by mammalian cells and bind receptors located on neighboring cells. They are a category of signaling molecules that are used extensively in inter-cellular communication. Cytokines have immunomodulatory functions and can promote the activation, inactivation, and/or differentiation of recipient cells.

“Cytokine receptor” is a class of receptors that bind cytokines. Type I cytokine receptors are defined by conserved motifs in their extracellular domain. An example of Type I cytokine receptor is the IL-2 receptor. Type II cytokine receptors bind the interferons. The Immunoglobulin (Ig) superfamily are ubiquitously expressed on several cells and tissues in vertebrates. The tumor necrosis receptor family members share a cysteine-rich extracellular domain and binds such cytokines as TNF, CD40L, BAFF, CD27 and CD30. The Chemokine receptors are G protein coupled receptors. The TGF beta receptors.

Interleukin 6 (IL-6) is a cytokine involved in the acute-phase response and the humoral immune response. IL-6 plays both positive and negative roles in cell proliferation, negative roles in the regulation of apoptosis and the negative roles in chemokine biosynthesis. IL-6 can be secreted by B cells, T cells and macrophages in response to tissue trauma, tissue damage or infection leading to inflammation. Additionally, IL-6 is produced by skeletal muscle in response to muscle contraction and in smooth muscle cells in the tunica media of many blood vessels in a pro-inflammatory response. IL-6 is secreted by osteoblasts to stimulate osteoclast formation. IL-6 can act in an autocrine and paracrine manner on B cells to modulate B cell activity and effector functions (for example, immunoglobulin production). IL-6 plays a role as an anti-inflammatory cytokine in specific instances, which is mediated through its effects on TNF and IL-1 and the activation of IL-1RA and IL-10. Local IL-6 levels can have different effects in the context of a tissue microenvironment. Systemic levels of IL-6 may not affect peripheral immune cell responses in tissue microenvironments. Additionally, systemic IL-6 levels have been implicated in conditions of autoimmune disease and bloodstream infection but the importance of which is unclear as healthy humans can have elevated systemic levels of IL-6. The alteration of IL-6 in the microenvironment can be advantageous in specific disease indications. Dysregulated production of IL-6 is involved in the pathogenesis of diseases such as multiple myeloma, autoimmune diseases and certain cancers.

“IL-6 Receptor” (IL-6R) is a protein complex consisting of the IL-6 receptor subunit alpha (IL6R alpha) and IL-6 signal transducer (gp130). IL-6R has been shown to interact with IL-6 and ciliary neurotrophic factor. The binding of IL-6 to IL-6R induces the subsequent homodimerization of the signal-transducing gp130. Dysregulation of IL-6R is involved in the pathogenesis of diseases such as multiple myeloma, autoimmune diseases and certain cancers.

Interleukin-10 (IL-10) is an immunomodulatory cytokine that can be upregulated in various types of cancer and has been demonstrated to be absent or suppressed in various autoimmune diseases. The biological role of IL-10 is complex but is positively correlated with metastatic growth, tumor aggression and the induction of immune tolerance or its suppression. IL-10 expression downregulates Th1 cytokines and induces T-regulatory responses. IL-10 production by regulatory B cells (B10 or Bregs), macrophage (M2), dendritic cells, regulatory T cells (Tregs) and other cell types has been demonstrated. IL-10 binds its cognate receptor, IL-10R, which can have numerous isotypes. IL-10 signaling can activate the JAK/STAT3 pathway to direct gene expression associated with homeostasis and healing response. The induction of IL-10 in a local setting can be advantageous in affecting peripheral immune cell responses in tissue microenvironments. The induction of IL-10 in a systemic manner can lead to systemic immune suppression and can be advantageous in specific disease indications. Mutations affecting IL-10, IL-10R and STAT3 have been demonstrated in various cancers and autoimmune diseases (eg, inflammatory bowel disease, systemic lupus erythematosus, rheumatoid arthritis, ankylosing spondylitis, Celiac disease and others).

TFGβ—Transforming growth factor-β (TGFβ) is a multifunctional cytokine, with important roles in homeostasis maintenance. TGFβ binds transmembrane serine/threonine kinase receptors to activate Smad transcriptional regulators. Alterations in TGFβ signaling have been implicated in cancer, fibrosis and systemic sclerosis. Elevated TGFβ levels positively correlate with disease severity. In cancer, TGFβ promotes tumor invasion and metastasis by affecting fibroblast activation and promoting immune suppression. TGFβ strongly promotes the production and deposition of extracellular matrix. The importance of TGFβ in immunoregulation and tolerance has been increasingly recognized. TGFβ has multiple suppressive actions on T cells, B cells, macrophages and other cells. Increased TGFβ production correlates with protection and/or recovery from autoimmune diseases. TGFβ can act in combination with other inhibitory molecules to maintain a state of tolerance. The induction of TGFβ in a local setting can be advantageous in affecting peripheral immune cell responses in tissue microenvironments. The induction of TGFβ in a systemic manner can lead to systemic immune suppression and can be advantageous in specific disease indications. Mutations in TGFβ signaling pathways have been demonstrated in allograft and transplant outcomes, lung, hepatocellular, breast and ovarian cancers, asthma, allergy and autoimmune disease.

“Protein kinase” is a phosphotransferase, an enzyme that transfers phosphate groups from high-energy donor molecules such as ATP, to specific substrates. Kinases act on and modify the activity of specific proteins. Kinases transmit signals and control complex processes in cells.

A review of the TNF receptor superfamily may be found in Baker and Reddy (1998) Oncogene 17(25):3261-70. Gravestein and Borst (1998) Semin Immunol 10(6):423-34 also review this receptor superfamily. The TNF superfamily is a group of cytokines with important functions in immunity and inflammation, and in the control of cell proliferation, differentiation, and apoptosis. TNFa is the founding member of the 19 different proteins so far identified within this family. Other signaling molecules of interest for the present invention include CD40 ligand (CD154) and BAFF. TNF family members exert their biological effects through the TNFR superfamily of cell surface receptors. Some of these receptors share a stretch of approximately 80 amino acids within their cytoplasmic region, the death domain (DD), which is critical for recruiting the death machinery after ligation of the receptors.

The mammalian TNFR family members are type I membrane proteins and, in spite of their low degree of homology (20±25%), are grouped together as such due to the presence of conserved cysteine residues in the extracellular ligand-binding domain. In general, each receptor contains varying numbers of cysteine rich domains (CRDs), each of which is characterized by the presence of approximately six cysteine residues that are interspersed within a stretch of 40 amino acids. The presence of the CRDs, based on available crystallographic data for TNFRI, has allowed this protein superfamily to add a different perspective from which these (as opposed to other) growth factor receptors are studied: a functional TNF superfamily receptor is typically a trimeric or multimeric complex which is stabilized via intracysteine disulfide bonds that are formed between the CRDs of individual subunit members.

“TNFR1” is constitutively expressed in most cell types, and multiple experimental approaches have confirmed that TNFR1 mediates majority of the biological effects attributed to its cognate ligand, TNF (TNF). The binding of TNF to TNFR1 triggers a series of intracellular events initiated by the recruitment of a key adaptor protein TNFR1-associated death domain protein (TRADD) to the receptor complex. Downstream of TRADD, two signaling complexes are formed, the plasma membrane-bound complex (complex I) consisting of TNFR1, TRADD, the receptor interacting protein (RIP), and TNF receptor-associated factor 2 (TRAF2), leading to rapid activation of NF-κB and the mitogen-activated protein kinases (MAPK) pathways, and 2) the cytoplasmic complex (complex II) containing TRADD, RIP, FAS-associated death domain protein (FADD), and caspase 8, essential for TNF-induced apoptosis through a caspase cascade. In most cells the combination of the above signaling pathways determines the diverse biological activities of TNF, including cell growth, development, oncogenesis, inflammation, stress-induced signaling, and cell death.

Treg. Regulatory T cells (Tregs) are characterized by the expression of the transcription factor Foxp3 and play a key role in immune homeostasis. Tregs function by the suppression of T cell immunity. One important aspect in Treg function is CTLA-4 expression to down-regulate activated antigen presenting cells in a cell-to-cell contact manner. Another mechanism contributing to the suppressive effects of Tregs is the production of IL-10, TGFβ, IL-35 and others. Low Treg levels are associated with spontaneous development of autoimmune disease, allergy and immunopathology and elevated Th1 and/or Th17 T cell populations. The development of Treg inducing therapeutics is needed.

Breg. Regulatory B cells (Bregs or B10) are specialized B cells characterized by the production of IL-10 cytokine. Bregs have been demonstrated to suppress immune responses and control various immunopathologies. Breg-mediated suppression is important in the maintenance of peripheral tolerance and is directly mediated by the production of IL-10 and/or TGFβ. In some cases, Bregs can also exert their regulatory effects by direct cell-to-cell contact with pathogenic T cells in conjunction with CD40 or B7 co-stimulatory molecules. Bregs have also been demonstrated to control dendritic cell functions (Lo-Man R. (2011) Immunother 3(4 Suppl):19-20). Breg development has been induced only through B cell receptor, CD40 and TLR9 activation (Lemione et al, (2009) Ann N Y Acad Sci 1173:260-267). Reduction in IL-10 producing B cells in multiple sclerosis is associated with increased disease. Additionally, a lack of IL-10 producing Bregs is associated with exacerbated autoimmune disease and an increased frequency of Th1 and/or Th17 T cells and a decrease in Tregs (Carter et al (2011) J Immunol 186:5569-5579). Methods to increase and/or activate the Breg population in individuals with autoimmune disease, asthma, allergy or transplanted tissue are needed.

Without being limited to theory, the data presented herein suggest that cardiac glycosides activate regulatory B cells, which express the anti-inflammatory cytokines IL-10 and TGFβ and regulate pro-inflammatory T cells.

Numerous biologic TNF antagonists such as adalimumab, infliximab, and etanercept have been developed which act extracellularly to inhibit TNF binding to TNFR, however the expense and patient response rate to these treatments are not optimal.

“Subject” or “patient” shall mean any animal, such as a human, non-human primate, mouse, rat, guinea pig or rabbit.

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular (mediated by antigen-specific T cells or their secretion products) response. An “immunogen” is capable of inducing an immunological response against itself on administration to a mammal or due to autoimmune disease.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and CloneTech.

The subject methods are used for prophylactic or therapeutic purposes. As used herein, the term “treating” is used to refer to both prevention of relapses, and treatment of pre-existing conditions. For example, the prevention of autoimmune disease may be accomplished by administration of the agent prior to development of a relapse. The treatment of ongoing disease, where the treatment stabilizes or improves the clinical symptoms of the patient, is of particular interest.

The invention provides methods for treating inflammatory diseases. Inflammatory diseases of interest include autoimmune and inflammatory conditions, including rheumatoid arthritis (RA), systemic lupus erythematosus (SLE), myasthenia gravis (MG), type I diabetes, psoriasis, Crohn's disease and the other autoimmune and inflammatory conditions described herein. Inflammatory conditions also include type II diabetes, Alzheimer's disease, osteoarthritis and other diseases associated with aberrant inflammatory responses. The methods of the invention comprise administering to the subject an effective amount of a cardiac glycoside through a route and dosing regimen that is effective to generate or activate regulatory B cells, resulting in suppression or prevention of disease initiation, including prevention of progression from a pre-clinical to a clinical stage of disease, and to inhibit progression, or relapses of disease.

The invention also provides methods for treating diseases associated with aberrant activity or activation of monocyte-lineage cells such as but not limited to dendritic cells, osteoblasts, and macrophage. Monocyte-lineage cell dysfunction is a major contributor to tissue damage in autoimmune diseases, osteoarthritis, atherosclerosis, Alzheimer's, and type II diabetes.

The invention also provides information on tumor necrosis family receptor members (TNFRs). TNFRs act upstream of NFκB to regulate genes involved in inflammatory responses, developmental processes, cellular growth and apoptosis. In addition, persistently active TNFR signaling and NFκB activity are hallmarks in disease states including cancer, arthritis, chronic inflammation, asthma, neurodegenerative diseases and heart disease. Members of this conserved transmembrane receptor family are constitutively expressed or induced on the surface of most cell types including but not limited to immune cells, endothelial, epithelial and stromal cells, Members include TNFR1, TNFR2, CD40, BAFFR, BCMA, TACI, 4-1BB, CD27, CD30, GITR, OX40, LTβR, RANK, and HVEM, FAS, CD137, CD120a, CD120b, lymphotoxin-beta receptor, CD134, TNFRSF6B, TNFRSF10A, TNFRSF10B, TNFRSF10C, TNFRSF10D, RANK, osteoprotegerin, TNFRSF12A, TNFRSF13B, TNFRSF13C, TNFRSF14, nerve growth factor receptor (NGFR), TNFRSF17, TNFRSF18, TNFRSF19, TNFRSF21, TNFRSF25 and ectodysplasin A2 receptor. Activation of TNFRs leads to downstream kinase activity, transcription activity and cellular responses such as proliferation, cytokine and chemokine secretion, cell adhesion molecule expression, metalloproteinase secretion and anti-apoptotic effector functions.

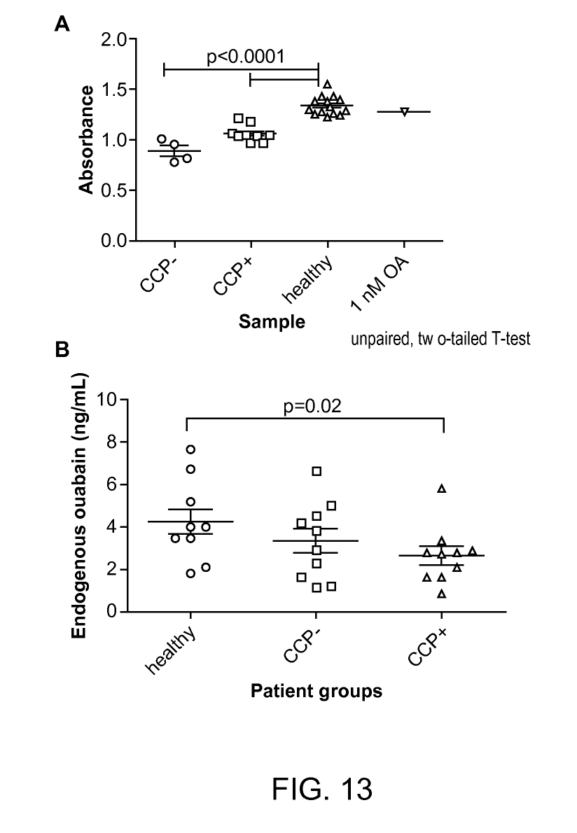

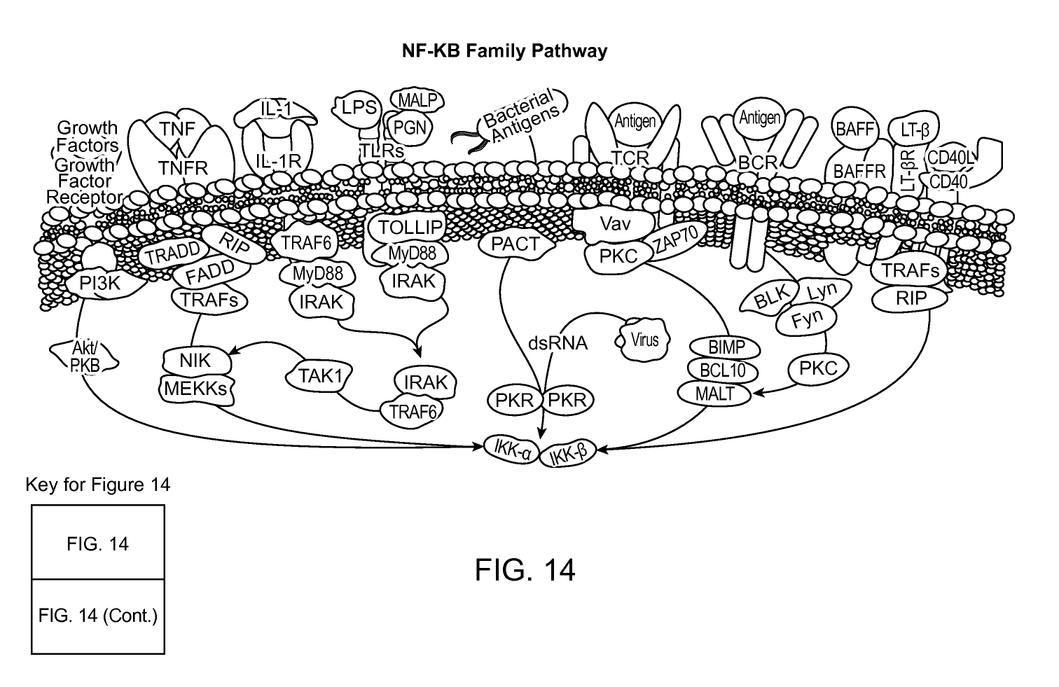

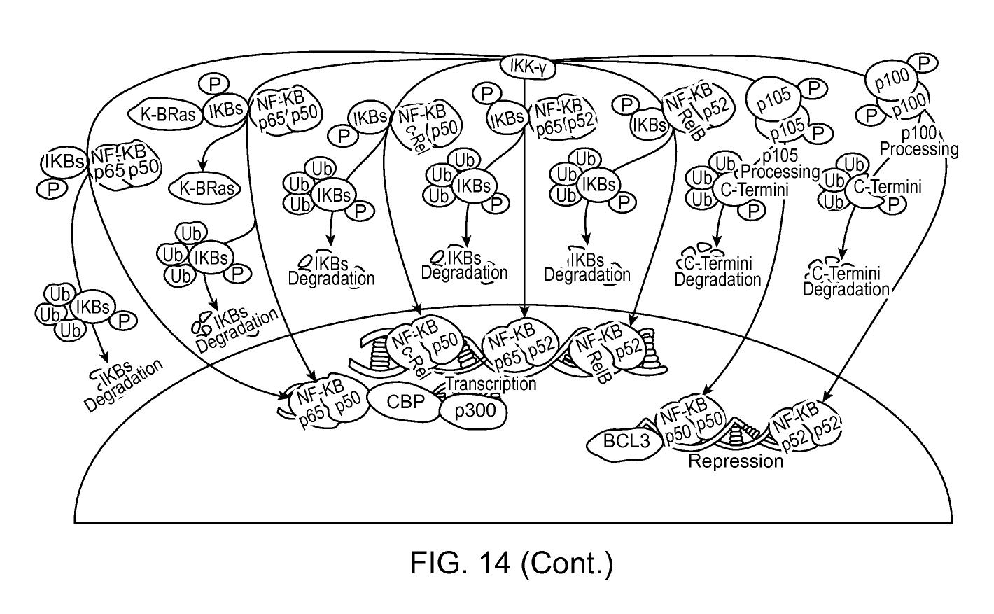

TNFR1 is a prototypical member of the TNFR family. TNFR1 is expressed on the surface of most mammalian cells. The binding of the ligand, TNF, to TNFR1 activates the receptor and causes the recruitment of adaptor molecules, including TRADD and others, to the intracellular domain of the receptor. Adaptor molecule activation causes downstream kinases to activate and results in the activation of transcription factors such as NFkB and AP-1 (FIG. 13).

“CD40” is a TNFR member that is constitutively expressed on monocytes, dendritic cells, macrophage and B cells. CD40 is induced on T cells, smooth muscle, epithelial and endothelial cells, platelets, fibroblast-like stromal cells, microglia and hepatocytes. The functional outcomes of CD40 ligand (CD40L) binding are cellular activation, CD40L expression, cytokine and chemokine responsiveness and expression, cell proliferation, immunoglobulin class switching (B cells) apoptosis rescue and adhesion molecule expression. CD40L mutations have been demonstrated to be a causal agent for hyper-IgM syndrome. CD40 knockout mice have severely reduced serum levels of IgG1 and IgE isotypes.

“BCMA” is a TNFR member that is expressed on B cells. Binding of the ligand BAFF to BCMA induces differentiation in later stage B cells. BMCA activation is also important for plasmablast survival.

“TACI” is a TNFR member that is expressed in B cells. TACI binding to the ligand BAFF induces B cells to mediate cytotoxic T cell priming.

“BAFFR” is a TNFR member that is expressed on B cells. Binding of the ligand, BAFF to BAFFR causes numerous B cell responses, BAFF/BAFFR interaction controls the activation of peripheral B cells. Deletion of BAFFR results in a significant loss of B cell survival (90%). BAFF activation triggers CD40L-independent immunoglobulin class switching. BAFF overexpression in mice leads to B cell hyperplasia and SLE-like symptoms and SLE patients have increased BAFF levels.

“Sublingual” refers to a method of administering dosage forms, which comprise cardiac glycosides administered to the mucosal surfaces in the mouth, including the mucosal surfaces under the tongue. It is shown herein to be an effective dosing regimen for cardiac glycosides, which diffuse efficiently into the blood stream through the mucosal membranes under the tongue.

“Endogenous cardiac glycoside reactivity” (ECGR) is a description of cardiac glycosides that have been detected, by ELISA or other antibody-based methods, to exist and to be synthesized in humans and other mammals.

### Conditions for Analysis and Therapy

The compositions and methods of the invention find use in combination with a variety of autoimmune conditions, which include, without limiting, the following conditions.

Rheumatoid Arthritis (RA). RA is a chronic syndrome characterized by usually symmetric inflammation of the peripheral joints, potentially resulting in progressive destruction of articular and periarticular structures, with or without generalized manifestations. The cause is unknown. A genetic predisposition has been identified and, in white populations, localized to a pentapeptide in the HLA-DR beta1 locus of class II histocompatibility genes. Environmental factors may also play a role. Immunologic changes may be initiated by multiple factors. About 0.6% of all populations are affected, women two to three times more often than men. Onset may be at any age, most often between 25 and 50 yr.

Prominent immunologic abnormalities that may be important in pathogenesis include immune complexes (antibody-antigen complexes) found in joint fluid derived from a subset of RA patients. Plasma cells produce antibodies that contribute to these complexes. Lymphocytes that infiltrate the synovial tissue are primarily T helper cells, which can produce pro-inflammatory cytokines. Macrophages and their cytokines (e.g., tumor necrosis factor, granulocyte-macrophage colony-stimulating factor) are also abundant in diseased synovium. Increased adhesion molecules contribute to inflammatory cell emigration and retention in the synovial tissue. Increased macrophage-derived lining cells are prominent along with some lymphocytes and vascular changes in early disease.

In chronically affected joints, the normally delicate synovium develops many villous folds and thickens because of increased numbers and size of synovial lining cells and colonization by lymphocytes and plasma cells. The lining cells produce various materials, including collagenase and stromelysin, which can contribute to cartilage destruction; matrix metalloproteinases, which are degradative enzymes; interleukin-1, which stimulates lymphocyte proliferation; and prostaglandins. The infiltrating cells, initially perivenular but later forming lymphoid follicles with germinal centers, synthesize interleukin-2, TNF, IL-6, and other cytokines, RF, and other immunoglobulins. Fibrin deposition, fibrosis, and necrosis are also are present. Hyperplastic synovial tissue (pannus) may erode cartilage, subchondral bone, articular capsule, and ligaments. Neutrophils are not prominent in the synovium but often predominate in the synovial fluid.

Onset is usually insidious, with progressive joint involvement, but may be abrupt, with simultaneous inflammation in multiple joints. Tenderness in nearly all inflamed joints is the most sensitive physical finding. Synovial joint tenderness and swelling eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint.

SLE. Systemic lupus erythematosus (SLE) is an autoimmune disease characterized by polyclonal B cell activation, which results in a variety of anti-protein and non-protein autoantibodies (see Kotzin et al. (1996) Cell 85:303-306 for a review of the disease). These autoantibodies form immune complexes that deposit in multiple organ systems, causing tissue damage. SLE is a difficult disease to study, having a variable disease course characterized by exacerbations and remissions. For example, some patients may demonstrate predominantly skin rash and joint pain, show spontaneous remissions, and require little medication. The other end of the spectrum includes patients who demonstrate severe and progressive kidney involvement (glomerulonephritis) that requires therapy with high doses of steroids and cytotoxic drugs such as cyclophosphamide.

Multiple factors may contribute to the development of SLE. Several genetic loci may contribute to susceptibility, including the histocompatibility antigens HLA-DR2 and HLA-DR3. The polygenic nature of this genetic predisposition, as well as the contribution of environmental factors, is suggested by a moderate concordance rate for identical twins, of between 25 and 60%.

Many causes have been suggested for the origin of autoantibody production. Proposed mechanisms of T cell help for anti-dsDNA antibody secretion include T cell recognition of DNA-associated protein antigens such as histones and recognition of anti-DNA antibody-derived peptides in the context of class II MHC. The class of antibody may also play a factor. In the hereditary lupus of NZB/NZW mice, cationic IgG2a anti-double-stranded (ds) DNA antibodies are pathogenic. The transition of autoantibody secretion from IgM to IgG in these animals occurs at the age of about six months, and T cells may play an important role in regulating the IgG production.

Disease manifestations result from recurrent vascular injury due to immune complex deposition, leukothrombosis, or thrombosis. Additionally, cytotoxic antibodies can mediate autoimmune hemolytic anemia and thrombocytopenia, while antibodies to specific cellular antigens can disrupt cellular function. An example of the latter is the association between anti-neuronal antibodies and neuropsychiatric SLE.

Myasthenia gravis: is a neuromuscular disease leading to fluctuating muscle weakness and fatiguability. It is an autoimmune disorder, in which weakness is caused by circulating antibodies that block acetylcholine receptors at the post-synaptic neuromuscular junction, inhibiting the stimulative effect of the neurotransmitter acetylcholine. Myasthenia is treated medically with cholinesterase inhibitors or immunosuppressants, and, in selected cases, thymectomy. At 200-400 cases per million it is one of the less common autoimmune disorders.

Psoriasis is a chronic skin disease, characterized by scaling and inflammation. Psoriasis affects 1.5 to 2 percent of the United States population, or almost 5 million people. It occurs in all age groups and about equally in men and women. People with psoriasis suffer discomfort, restricted motion of joints, and emotional distress. When psoriasis develops, patches of skin thicken, redden, and become covered with silvery scales, referred to as plaques. Psoriasis most often occurs on the elbows, knees, scalp, lower back, face, palms, and soles of the feet. The disease also may affect the fingernails, toenails, and the soft tissues inside the mouth and genitalia. About 10 percent of people with psoriasis have joint inflammation that produces symptoms of arthritis.

When skin is wounded, a wound healing program is triggered, also known as regenerative maturation. Lesional psoriasis is characterized by cell growth in this alternate growth program. In many ways, psoriatic skin is similar to skin healing from a wound or reacting to a stimulus such as infection, where the keratinocytes switch from the normal growth program to regenerative maturation. Cells are created and pushed to the surface in as little as 2-4 days, and the skin cannot shed the cells fast enough. The excessive skin cells build up and form elevated, scaly lesions. The white scale (called “plaque”) that usually covers the lesion is composed of dead skin cells, and the redness of the lesion is caused by increased blood supply to the area of rapidly dividing skin cells.

The exact cause of psoriasis in humans is not known, although it is generally accepted that it has a genetic component, and a recent study has established that it has an autoimmune component. Whether a person actually develops psoriasis is hypothesized to depend on something “triggering” its appearance. Examples of potential “trigger factors” include systemic infections, injury to the skin (the Koebner phenomenon), vaccinations, certain medications, and intramuscular injections or oral steroid medications. The chronic skin inflammation of psoriasis is associated with hyperplastic epidermal keratinocytes and infiltrating mononuclear cells, including CD4+ memory T cells, neutrophils and macrophages.

Diabetes Mellitus (DM) is a syndrome characterized by hyperglycemia resulting from absolute or relative impairment in insulin secretion and/or insulin action. Although it may occur at any age, type I diabetes mellitus (T1D) most commonly develops in childhood or adolescence and is the predominant type of DM diagnosed before age 30. This type of diabetes accounts for 10 to 15% of all cases of DM and is characterized clinically by hyperglycemia and a propensity to DKA. The pancreas produces little or no insulin.

About 80% of patients with T1D have specific HLA phenotypes associated with detectable serum islet cell cytoplasmic antibodies and islet cell surface antibodies (antibodies to glutamic acid decarboxylase (GAD) and to insulin are found in a similar proportion of cases). In these patients, T1D results from a genetically susceptible, immune-mediated, selective destruction of >90% of their insulin-secreting beta cells. Their pancreatic islets exhibit insulitis, which is characterized by an infiltration of T lymphocytes accompanied by macrophages and B lymphocytes and by the loss of most of the beta cells, without involvement of the glucagon-secreting alpha cells. Cell-mediated immune mechanisms are believed to play the major role in the beta-cell destruction. The antibodies present at diagnosis usually become undetectable after a few years. They may be primarily a response to beta-cell destruction, but some are cytotoxic for beta cells and may contribute to their loss. The clinical onset of T1D may occur in some patients years after the insidious onset of the underlying autoimmune process.

In white populations, a strong association exists between T1D diagnosed before age 30 and specific HLA-D phenotypes (HLA-DR3, HLA-DR4, and HLA-DR3/HLA-DR4). One or more genes that convey susceptibility to T1D are believed to be located near or in the HLA-D locus on chromosome 6. Specific HLA-DQ alleles appear to be more intimately related to risks for or protection from T1D than HLA-D antigens, and evidence suggests that genetic susceptibility to type T1D is probably polygenic. Only 10 to 12% of newly diagnosed children with T1D have a first-degree relative with T1D, and the concordance rate for T1D in monozygotic twins is <=50%. Thus, in addition to the genetic background, environmental factors affect the appearance of T1D. Such environmental factors may be viruses (congenital rubella, mumps, and coxsackie B viruses may incite the development of autoimmune beta-cell destruction) and exposure to cow's milk rather than maternal milk in infancy (a specific sequence of albumin from cow's milk may cross-react with islet protein). Geography may play a role in exposure, as the incidence of T1D is particularly high in Finnland and Sardinia.

Recently, type 2 diabetes (T2D) (formerly called non-insulin-dependent diabetes mellitus (NIDDM), or adult-onset diabetes) has recently been recognized as an inflammatory disorder involving macrophage producing inflammatory cytokines. T2D is a disorder that is characterized by high blood glucose in the context of insulin resistance and relative insulin deficiency. While it is often initially managed by increasing exercise and dietary modification, medications are typically needed as the disease progresses. Over 5% of the U.S. population has T2D. Although traditionally considered a disease of adults, T2D is increasingly diagnosed in children in parallel to rising obesity rates. Recently, it has been recognized that low-grade tissue inflammation, arising from obesity, contributes to insulin resistance, the major cause of T2D. Further, inflammatory macrophage and their production of inflammatory mediators have been shown to play a central role in mediating T2D.

Multiple sclerosis: Multiple sclerosis (MS) is a debilitating, inflammatory, neurological illness characterized by demyelination of the central nervous system. The disease primarily affects young adults with a higher incidence in females. Symptoms of the disease include fatigue, numbness, tremor, tingling, dysesthesias, visual disturbances, dizziness, cognitive impairment, urological dysfunction, decreased mobility, and depression. Four types classify the clinical patterns of the disease: relapsing-remitting, secondary progressive, primary-progressive and progressive-relapsing (S. L. Hauser and D. E. Goodkin, Multiple Sclerosis and Other Demyelinating Diseases in Harrison's Principles of Internal Medicine 14th Edition, vol. 2, Mc Graw-Hill, 1998, pp. 2409-19).

Systemic sclerosis: Systemic sclerosis (SSc, or scleroderma) is an autoimmune disease characterized by fibrosis of the skin and internal organs and widespread vasculopathy. Patients with SSc are classified according to the extent of cutaneous sclerosis: patients with limited SSc have skin thickening of the face, neck, and distal extremities, while those with diffuse SSc have involvement of the trunk, abdomen, and proximal extremities as well. Internal organ involvement tends to occur earlier in the course of disease in patients with diffuse compared with limited disease (Laing et al. (1997) Arthritis. Rheum. 40:734-42). The majority of patients with diffuse SSc who develop severe internal organ involvement will do so within the first three years after diagnosis at the same time the skin becomes progressively fibrotic (Steen and Medsger (2000) Arthritis Rheum. 43:2437-44). Common manifestations of diffuse SSc that are responsible for substantial morbidity and mortality include interstitial lung disease (ILD), Raynaud's phenomenon and digital ulcerations, pulmonary arterial hypertension (PAH) (Trad et al. (2006) Arthritis. Rheum. 54:184-91), musculoskeletal symptoms, and heart and kidney involvement (Ostojic and Damjanov (2006) Clin. Rheumatol. 25:453-7). Current therapies focus on treating specific symptoms, but disease-modifying agents targeting the underlying pathogenesis are lacking.

Autoimmune Uveitis. Autoimmune uveitis is an autoimmune disease of the eye that is estimated to affect 400,000 people, with an incidence of 43,000 new cases per year in the U.S. Autoimmune uveitis is currently treated with steroids, immunosuppressive agents such as methotrexate and cyclosporin, intravenous immunoglobulin, and TNF-antagonists.

Experimental autoimmune uveitis (EAU) is a T cell-mediated autoimmune disease that targets neural retina, uvea, and related tissues in the eye. EAU shares many clinical and immunological features with human autoimmune uveitis, and is induced by peripheral administration of uveitogenic peptide emulsified in Complete Freund's Adjuvant (CFA).

Self-proteins targeted by the autoimmune response in human autoimmune uveitis may include S-antigen, interphotoreceptor retinoid binding protein (IRBP), rhodopsin, and recoverin.

Primary Billiary Cirrhosis. Primary Biliary Cirrhosis (PBC) is an organ-specific autoimmune disease that predominantly affects women between 40-60 years of age. The prevalence reported among this group approaches 1 per 1,000. PBC is characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjögren's Syndrome, CREST Syndrome, Autoimmune Thyroid Disease and Rheumatoid Arthritis. Attention regarding the driving antigen(s) has focused on the mitochondria for over 50 years, leading to the discovery of the antimitochondrial antibody (AMA) (Gershwin et al., Immunol Rev 174:210-225, 2000); (Mackay et al., Immunol Rev 174:226-237, 2000). AMA soon became a cornerstone for laboratory diagnosis of PBC, present in serum of 90-95% patients long before clinical symptoms appear. Autoantigenic reactivities in the mitochondria were designated as M1 and M2. M2 reactivity is directed against a family of components of 48-74 kDa. M2 represents multiple autoantigenic subunits of enzymes of the 2-oxoacid dehydrogenase complex (2-OADC) and is another example of the self-protein, -polypeptide, or -peptide of the instant invention. Studies identifying the role of pyruvate dehydrogenase complex (PDC) antigens in the etiopathogenesis of PBC support the concept that PDC plays a central role in the induction of the disease (Gershwin et al., Immunol Rev 174:210-225, 2000); (Mackay et al., Immunol Rev 174:226-237, 2000). The most frequent reactivity in 95% of cases of PBC is the E2 74 kDa subunit, belonging to the PDC-E2. There exist related but distinct complexes including: 2-oxoglutarate dehydrogenase complex (OGDC) and branched-chain (BC) 2-OADC. Three constituent enzymes (E1,2,3) contribute to the catalytic function which is to transform the 2-oxoacid substrate to acyl co-enzyme A (CoA), with reduction of NAD⁺ to NADH. Mammalian PDC contains an additional component, termed protein X or E-3 Binding protein (E3BP). In PBC patients, the major antigenic response is directed against PDC-E2 and E3BP. The E2 polypeptide contains two tandemly repeated lipoyl domains, while E3BP has a single lipoyl domain. PBC is treated with glucocorticoids and immunosuppressive agents including methotrexate and cyclosporin A.

A murine model of experimental autoimmune cholangitis (EAC) uses intraperitoneal (i.p.) sensitization with mammalian PDC in female SJL/J mice, inducing non-suppurative destructive cholangitis (NSDC) and production of AMA (Jones, J Clin Pathol 53:813-21, 2000).

Inflammatory bowel diseases: Inflammatory bowel diseases, include Crohn's disease and ulcerative colitis, involve autoimmune attack of the bowel. These diseases cause chronic diarrhea, frequently bloody, as well as symptoms of colonic dysfunction.

Additional examples of autoimmune diseases include those involving the thyroid (Grave's disease and Hashimoto's thyroiditis), peripheral nerves (Guillain-Barre Syndrome and other autoimmune peripheral neuropathies), the CNS (acute disseminated encephalomyelitis, ADEM), the skin (pemphigoid (bullous), pemphigus foliaceus, pemphigus vulgaris, coeliac sprue-dermatitis, vitiligo), the liver and gastrointestinal system (primary biliary cirrhosis, pernicious anemia, autoimmune hepatitis, autoimmune gastritis, celiac disease [autoimmunity against transglutaminase and various native and cleaved glutens]), and the eye (autoimmune uveitis). There are also multiple “autoimmune rheumatic diseases” (Sjögren's syndrome, discoid lupus, antiphospholipid syndrome, CREST, mixed connective tissue disease (MCTD), polymyositis and dermatomyositis, and Wegener's granulomatosus).

TABLE 1
Examples of Autoimmune Diseases and their Target Autoantigens.

| | | Self-Protein(s) Associated With An |
|---|---|---|
| Autoimmune Disease | Tissue Targeted | Autoimmune Disease |
| Multiple sclerosis | central nervous | myelin basic protein, proteolipid protein, |
| | system | myelin associated glycoprotein, cyclic |
| | | nucleotide phosphodiesterase, yelin- |
| | | associated glycoprotein, myelin-associated |
| | | oligodendrocytic basic protein; alpha-B- |
| | | crystalin |
| Guillian Barre | peripheral nerv. | peripheral myelin protein I and others |
| Syndrome | sys. | |
| Insulin Dependent | b cells in islets of | tyrosine phosphatase IA2, IA-2b; glutamic |
| Diabetes Mellitus | pancreas | acid decarboxylase (65 and 67 kDa forms), |
| | | carboxypeptidase H, insulin, proinsulin, heat |
| | | shock proteins, glima 38, islet cell antigen 69 KDa, |
| | | p52, ganglioside antigens, islet cell |
| | | glucose transporter GLUT-2 |
| Rheumatoid Arthritis | synovial joints | Immunoglobulin, fibrin, filaggrin, type I, II, III, |
| | | IV, V, IX, and XI collagens, GP-39, hnRNPs |
| Autoimmune Uveitis | eye, uvea | S-antigen, interphotoreceptor retinoid binding |
| | | protein (IRBP), rhodopsin, recoverin |
| Primary Biliary Cirrhosis | biliary tree of liver | pyruvate dehydrogenase complexes (2- |
| | | oxoacid dehydrogenase) |
| Autoimmune Hepatitis | Liver | Hepatocyte antigens, cytochrome P450 |
| Pemphigus vulgaris | Skin | Desmoglein-1, -3, and others |
| Myasthenia Gravis | nerve-muscle junct. | acetylcholine receptor |
| Autoimmune gastritis | stomach/parietal | H⁺/K⁺ ATPase, intrinsic factor |
| | cells | |
| Pernicious Anemia | Stomach | intrinsic factor |
| Crohn's disease | Intestine | Flagellin |
| Vasculitis | Blood vessels and | |
| | other tissues | |
| Polymyositis | Muscle | histidyl tRNA synthetase, other synthetases, |
| | | other nuclear antigens |
| Autoimmune Thyroiditis | Thyroid | Thyroglobulin, thyroid peroxidase |
| Graves's Disease | Thyroid | Thyroid-stimulating hormone receptor |
| Psoriasis | Skin | Unknown |
| Vitiligo | Skin | Tyrosinase, tyrosinase-related protein-2 |
| Systemic Lupus Eryth. | Systemic | nuclear antigens: DNA, histones, |
| | | ribonucleoproteins |
| Celiac Disease | Small bowel | Transglutaminase |

Other diseases arising from dysregulation of inflammatory responses. In recent years, several chronic diseases have been demonstrated to in part arise from dysregulated inflammatory responses. Such diseases include type 2 diabetes (T2D), Alzheimer's disease, macular degeneration, osteoarthritis and other diseases.

Alzheimer's disease. Alzheimer's disease and other neurodegenerative diseases are a category of diseases of the central nervous system for which there is increasing evidence for an inflammatory etiology. All are characterized by a slowly progressive destruction or degeneration of nerve cells (Temlett, Curr Opin Neurol 9, 303-7, 1996); (Dickson, Curr Opin Neurol 14, 423-32, 2001); (Kaye, Neurology 51, S45-52; discussion S65-7, 1998); (Prusiner, Proc Natl Acad Sci USA 95, 13363-83, 1998); (Cummings et al., Neurology 51, S2-17; discussion S65-7, 1998); (Lin et al., Neuron 24, 499-502, 1999); (Chesebro, Neuron 24, 503-6, 1999); (Ross, Neuron 19, 1147-50, 1997); (Yankner, Neuron 16, 921-32, 1996); (Selkoe, Neuron 6, 487-98, 1991). The degeneration of neurons in the brain or spinal cord leads to devastating permanent clinical symptoms including in some cases profound dementia, abnormal movements, tremor, gait ataxia, or epileptiform activity. Common to nearly all of the neurodegenerative diseases is the progressive dementia which can manifest itself as a complete inability to care for oneself and a total lack of recognition of friends and family.

Another common feature of these diseases is the lack of an effective therapy for any of them. Most of the treatments available today focus on supportive care of the late symptoms and none are directed at the underlying pathophysiologic causes of these diseases. For example, for Parkinson's disease medications are directed at and are usually effective in temporarily controlling the tremor associated with the disease, but no medications are effective in halting the progressive dementia and destruction of neurons within the substantia nigra of the brain (Jankovic, Neurology 55, S2-6, 2000). As another example, in Alzheimer's disease until recently no treatments were available for the progressive dementia that characterizes this disease. Several cholinesterase inhibitors have now been approved for use in Alzheimer's disease (Farlow and Evans, Neurology 51, S36-44; discussion S65-7, 1998) (Hake, Cleve Clin J Med 68, 608-9, 613-4, 616, 2001). These drugs presumably increase the amount of the neurotransmitter acetylcholine available in the brain, leading to improved function of those particular neurons that use acetylcholine as a transmitter. All of these drugs, as a whole, show only miniscule efficacy in clinical trials with the primary endpoint being improvement in cognitive testing. These drugs are also not directed at the primary pathophysiology of Alzheimer's disease, namely the destruction of the cholinergic neurons within the brain. Therefore, no current therapy aimed at the primary pathologic cause exists for any of the neurodegenerative diseases.

The majority of neurodegenerative disease also have in common the finding of aggregated or accumulated substances within the areas of the central nervous system that are most affected by the degenerative process. These abnormal accumulations, that can be found either extra- or intra-cellularly, may contribute to the death and destruction of the relevant neurons. Furthermore, the features and composition of the accumulations are specific for a particular disease. For example, the aggregates in Alzheimer's disease consist of a protein called amyloid beta, whereas for Parkinson's disease they are composed of a protein called alpha-synuclein (Dickson, Curr Opin Neurol 14:423-432, 2001); (Cummings et al., Neurology 51, S2-17; discussion S65-7, 1998). The neurodegenerative diseases characterized by the development and accumulation of such aggregates include Alzheimer's disease, Parkinson's disease, Huntington's disease, and prion disease (Yankner, Neuron 16:921-32, 1996); (Ross, Neuron 19:1147-50, 1997) (Chesebro, Neuron 24:503-506, 1999); (Dickson, Curr Opin Neurol 14:423-32, 2001).

Osteoarthritis. Osteoarthritis (OA) is the most common joint disorder in the world. The morbidity and health costs attributed to OA are substantial, and are anticipated to increase as the population ages. Although the joint is a complex structure comprised of multiple tissues that are perturbed in OA, the central lesion in all cases appears to be breakdown of the articular cartilage. This deterioration leads both directly and indirectly to pain and dysfunction, and atrophy of surrounding muscles often follows, which results in a decrease in mobility. The medical arts recognize two types of OA, idiopathic (primary) OA and secondary OA. A more aggressive form of OA, termed “erosive OA”, affects a small percentage of patients. Erosive OA is characterized clinically by extensive erosive and osteophytic changes at the distal interphalangeal and proximal interphalageal joints, and can also involve other joints. Low-grade inflammation is present in the vast majority of OA patients, and there is growing evidence that inflammatory responses involving monocyte-lineage cells and other inflammatory cell types may contribute to the pathogenesis of OA.

Patient outcomes for any of the above diseases, e.g. patient outcome following treatment by the methods of the invention, may be assessed using imaging-based criteria such as radiographic scores, clinical and laboratory criteria. Multiple different imaging, clinical and laboratory criteria and scoring systems have been and are being developed to assess disease activity and response to therapy in rheumatoid arthritis, systemic lupus erythmatosus, Crohn's disease, and many other autoimmune diseases.

In rheumatoid arthritis, response to therapy is conventionally measured using the American College of Rheumatology (ACR) Criteria. The ACR response criteria are a composite score comprising clinical (swollen joint count, tender joint count, physician and patient response assessment, and health assessment questionnaire), and laboratory (acute phase response) parameters; level of improvement is reported as an ACR20 (20%), ACR50 (50%) or ACR70 (70%) response, which indicates percent change (improvement) from the baseline score. A number of clinical trails based on which the anti-TNF agents infliximab (Remicade™), etanercept (Enbrel™) and adalimumab (Humira™) were approved to treat human RA utilized ACR response rates as a primary outcome measure.

Responses in rheumatoid arthritis many also be assessed using other response criteria, such as the Disease Activity Score (DAS), which takes into account both the degree of improvement and the patient's current situation. The DAS has been shown to be comparable in validity to the ACR response criteria in clinical trials. The definitions of satisfactory and unsatisfactory response, in accordance with the original DAS and DAS28. The DAS28 is an index consisting of a 28 tender joint count, a 28 swollen joint count, ESR (or CRP), and an optional general health assessment on a visual analogue scale (range 0-100) (Clinical and Experimental Rheumatology, 23(Suppl. 39):S93-99, 2005). DAS28 scores are being used for quantification of response mostly in European trials of (early) rheumatoid arthritis such as the COBRA or BeST studies.

Radiographic measures for response in RA include both conventional X-rays (plain films), and more recently magnetic resonance (MR) imaging, computed tomography (CT), ultrasound and other imaging modalities are being utilized to monitor RA patients for disease progression. Such techniques are used to evaluate patients for inflammation (synovitis), joint effusions, cartilage damage, bony erosions and other evidence of joint damage. Methotrexate, anti-TNF agents and DMARD combinations have been demonstrated to reduce development of bony erosions and other measures of joint inflammation and destruction in RA patients. In certain cases, such as with anti-TNF agents, healing of bony erosions has been observed.

For response to therapy in systemic lupus erythematosus there exist a variety of scoring systems including the Ropes system, the National Institutes of Health [NIH] system, the New York Hospital for Special Surgery system, the British Isles Lupus Assessment Group [BILAG] scale, the University of Toronto SLE Disease Activity Index [SLE-DAI], and the Systemic Lupus Activity Measure [SLAM] (Arthritis and Rheumatism, 32(9):1107-18, 1989). The BILAG assessment consists of 86 questions; some based on the patient's history, some on examination findings and others on laboratory results. The questions are grouped under eight headings: General (Gen), Mucocutaneous (Muc), Neurological (Cns), Musculoskeletal (Msk), Cardiovascular and Respiratory (Car), Vasculitis (Vas), Renal (Ren), and Haematological (Hae). Based on the answers, a clinical score is calculated. The SLEDAI is a weighted, cumulative index of lupus disease activity.

The methods of the invention also provide for combination therapy, where the combination may provide for additive or synergistic benefits. Combinations of a cardiac glycoside may be obtained with a second agent selected from one or more of the general classes of drugs commonly used in the non-antigen specific treatment of autoimmune disease, which include corticosteroids and disease modifying drugs; or from an antigen-specific agent. These agents include methotrexate, leflunomide (Arava™), etanercept (Enbrel™), infliximab (Remicade™), adalimumab (Humira™), anakinra (Kineret™) rituximab (Rituxan™), CTLA4-Ig (abatacept), antimalarials, gold salts, sulfasalazine, d-penicillamine, cyclosporin A, cyclophosphamide azathioprine; and the like. Of particular interest are combinations with drugs targeting TNF, e.g. etanercept (Enbrel™), infliximab (Remicade™), adalimumab (Humira™). Combination of such drugs with cardiac glycosides allows a more sparing use of the biological agents.

Corticosteroids, e.g. prednisone, methylpredisone, prednisolone, solumedrol, etc. have both anti-inflammatory and immunoregulatory activity. They can be given systemically or can be injected locally. Corticosteroids are useful in early disease as temporary adjunctive therapy while waiting for disease modifying agents to exert their effects. Corticosteroids are also useful as chronic adjunctive therapy in patients with severe disease.

Disease modifying anti-rheumatoid drugs, or DMARDs have been shown to alter the disease course and improve radiographic outcomes in RA. It will be understood by those of skill in the art that these drugs are also used in the treatment of other autoimmune diseases.

Methotrexate (MTX) is a frequent first-line agent because of its early onset of action (4-6 weeks), good efficacy, favorable toxicity profile, ease of administration, and low cost. MTX is the only conventional DMARD agent in which the majority of patients continue on therapy after 5 years. MTX is effective in reducing the signs and symptorheumatoid arthritis of RA, as well as slowing or halting radiographic damage. Although the immunosuppressive and cytotoxic effects of MTX are in part due to the inhibition of dihydrofolate reductase, the anti-inflammatory effects in rheumatoid arthritis appear to be related at least in part to interruption of adenosine and TNF pathways. The onset of action is 4 to 6 weeks, with 70% of patients having some response. A trial of 3 to 6 months is suggested.

### Pharmaceutical Compositions

Cardiac glycosides serve as the active ingredient in pharmaceutical compositions formulated for the treatment of various disorders as described above, and include the use of currently available medications: Acetyldigitoxin, Allocar, Corramedan, Crystodigin, Digimerck, Digitoximun, Digitalysat, Acocantherin, Astrobain, G-Strophicor, Gratibain, Gratus Strophanthin, Kombetin, Purostrophan, Rectobaina, Solufantina, Lanatoside A and Lanatoside C, Bufalin, Telocinobufagenin, Marinobufagin, Marinobufagenin, Digitoxigenin, Digoxigenin, Gitaligenin, Ouabagenin, G-strophanthidin, Digoxin, Laoxin, Gitoxigenin, Oleandrin, Oleandrigenin, Strodival, Strophalen, Strophantinum G, Strophoperm, Strophanthidin, Strophanthin K, Strophosan, Strophosid, Uabaina, Uabanin, Cardiovite, Proscillan, Proslladin, Purgoxin, Sandoscill, Scillacrist, Caradrin, Caradrine, Carmazon, Deslanoside, Dihydroouabain, Procardin, Procilan, Prostosin, Protasin, Solestril, Stellarid, Scillarenin, Scillaridin A, Talucard, Tradenal, Wirnesin, Cardion, Coratol, Proszin, Talusin and Urgilan.

Plant, animal and synthetically-derived cardiac glycosides can be purified and reconstituted in oral, inhalation, injectable and transdermal formulations. Digitoxin and similar cardiac glycosides have >90% oral bioavailability, whereas ouabain and proscillaridin A have higher bioavailability with injectable formulations. The plants that cardiac glycosides have been identified in and isolated from include: Digitalis spp., Rhododendrons, Nerium oleander, Helleborus niger, Convallaria spp, Asclepias spp., Apocynum spp., Adenosma grandiforum, Acocanthera schimperi, Strophanthus spp., Urginea spp. and numerous other plant species listed in Table 1 and Table 2 (Hollman, 1995).

TABLE 1
Botanical sources and major chemical components of cardiac glycosides of clinical importance

| | | | | | |
|---|---|---|---|---|---|
| | | Split off by | | | |
| | | enzymatic | | | |
| | | and mild | | | |
| Plant | Precursor | alkaline | | Split off by | |
| source | glycoside | hydrolysis* | Glycoside | acid hydrolysis* | Aglycone or genin |

Digitalis

| | | | | | |
|---|---|---|---|---|---|
| D purpurea | Purpurea-glycoside A | Glucose | Digitoxin | Digitoxose (3) | Digitoxigenin |
| (leaf) | (deacetyldigilanid A) | | | | |
| | Purpurea-glycoside B | Glucose | Gitoxin | Digitoxose (3) | Gitoxigenin |
| | (deacetyldigilanid B) | | | | |
| | — | — | Gitalin | Digitoxose (2) | Gitaligenin |
| | | | | | (gitoxigenin hydrate) |
| D lanata | Lanatoside A | Glucose + | Digitoxin | Digitoxose (3) | Digitoxigenin |
| (leaf) | (digilanid A) | acetic acid | | | |
| | Lanatoside B | Glucose + | Gitoxin | Digitoxose (3) | Gitoxigenin |
| | (digilanid B) | acetic acid | | | |
| | Lanatoside C | Glucose + | Digoxin | Digitoxose (3) | Digoxigenin |
| | (digilanid C; | acetic acid | | | |
| | cedilanid) | | | | |

Strophanthus

| | | | | | |
|---|---|---|---|---|---|
| S kombe | K-strophanthoside | Glucose | K-strophanthin-β | Glucose + | Strophanthidin |
| (seed) | | | (strophanthin) | cymarose | |
| | K-strophanthoside | Glucose (2) | Cymarin | Cymarose | Strophanthidin |
| | K-strophanthin-β | Glucose | Cymarin | Cymarose | Strophanthidin |
| | — | — | Cymarol | Cymarose | Strophanthidol |
| S gratus | — | — | Ouabain | Rhamnose | Ouabagenin |
| (seed) | | | (G-strophanthin) | | (G-strophanthidin) |

Scilla (squill)

| | | | | | |
|---|---|---|---|---|---|
| Urginea | Scillaren A | Glucose | Proscillaridin A | Rhamnose | Scillaridin A |
| maritima or | | | | | |
| indica (bulb) | | | | | |

*One mole of sugar or acetic acid is split off, unless the number of moles is otherwise indicated in parentheses. (From The Pharmacological Basis of Therapeutics, Goodman I.S, Gilman A. 5th ed. Macmillan, New York, 1975. Reproduced by permission of the authors and publisher.)

TABLE 2
Families and genera containing cardiac glycosides

APOCYANACEAE
Acokanthera
Adenium
Apocyanum
Carissa
Cerbera
Nerium
Strophanthus
Tanghinia
Thecetia
ASCLEPIADACEAE
Atelepias
Calotropis
Cryptostegia
Gomphocarpus
Menabea
Pachyearpus
Periploca
Xysmalobium
CELASTRACEAE
Euonymus
CRUCIFERAE
Cheiranthus
Erysimum
LILIACEAE
Bouica
Convallaria
Ornithagalum
Rohdea
Urginea
MORACEAE
Antiaris
Antiaropsis
Castilla
Ogcodeia
LEGUMINOSAE
Coronilla
RANUNCULACEAE
Adonis
Helleborus
SCROPHULARIACEAE
Digitalis
Isoplexis
STERCULIACEAE
Mansonia
TILIACEAE
Corchorus

Dosing regimens in humans vary according to the cardiac glycoside and constitution thereof. Table 3 depicts representative dosing regimens reported in the literature compared to experimental regiments used in mice that are representative of human dosing. Successful dosing detailed in this application includes but is not limited to 0.03-0.4 mg/kg., or from 0.001 to 5 mg/kg.

TABLE 3
Dosing Regimen and Corresponding Serum Concentrations Obtained

| | | Dose given | | Serum conc. | |
|---|---|---|---|---|---|
| Organism | Drug | (per diem) | | (ng/mL) | Reference |
| Human | Digitoxin | 0.1 | mg/kg, oral | 17 | Smith, TW (1970) |
| | Ouabain | 0.25 | mg/kg, i.v. | 0.51 | Selden, et al. (1972) |
| | Proscillaridin | 3 × 0.5 | mg/kg, oral | 0.75 | Andersson, et al. (1975) |
| Mouse | Digitoxin | 0.3 | mg/kg, i.p. | 19 | Experimentally |
| | Ouabain | 0.2 | mg/kg, i.p. | 16 | determined |
| | Proscillaridin | 0.25 | mg/kg, i.p. | 21 | |

The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers such as PEG or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

Further guidance regarding formulations that are suitable for various types of administration can be found in Remington's. Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via sublingual, buccal, lingual, transmucosal, oral, intranasal, rectal, topical, intraperitoneal, intravenous, intramuscular, subcutaneous, subdermal, transdermal, intrathecal, or intracranial method.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

For sublingual administration and administration of cardiac glycosides to the mucosal surfaces of the mouth, the active ingredient, alone, or in combination with other suitable components can be made into sublingual formulations such as liquids, capsules, fast-dissolving tablets, capsules, powders, gels, thin strips and lozenges. In one embodiment, cardiac glycosides are contained in a fast-dissolving thin strip of dehydrated polymer to act as an excipient, such as cellulose, gelatin or starch that, when hydrated under the tongue with saliva can dissolve to release the cardiac glycoside(s) so they are quickly absorbed into the body via the blood vessels located in the mucosal surfaces. In another embodiment, cardiac glycosides are dissolved in a fast-dissolving lozenge composed of liquid paraffin, sugars or sugar-substitutes, non-crystallizing sorbitol solution or the like. Lozenges dissolve by mouth saliva to release cardiac glycosides for quick absorption by blood vasculature in the mucosal surfaces of the mouth.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which are composed of the packaged active ingredient with a suppository base. Suitable suppository bases include natural or synthetic triglycerides or paraffin hydrocarbons. In addition, it is also possible to use gelatin rectal capsules, which are composed of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostatics, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are preferably sterile. To the extent that a given compound must be synthesized prior to use, the resulting product is preferably substantially free of any potentially toxic agents, such as any endotoxins, which may be present during the synthesis or purification process. Compositions for parental administration are also preferably sterile, substantially isotonic and made under GMP conditions.

### Methods of Treatment

The cardiac glycoside compositions may be administered in a single dose, or in multiple doses, usually multiple doses over a period of time, e.g. daily, every-other day, weekly, semi-weekly, monthly etc. for a period of time sufficient to reduce severity of the inflammatory disease, which may comprise 1, 2, 3, 4, 6, 10, or more doses. In some embodiments of the invention a dosing every 2 days, every 4 days, more preferably every 3 days is used.

Determining a therapeutically or prophylactically effective amount of an agent can be done based on animal data using routine computational methods. In one embodiment, the therapeutically or prophylactically effective amount contains between 0.00001-1000 mg/kg patient weight. In another embodiment, the effective amount contains between about 0.001-10 mg/kg patient weight, as applicable. In a further embodiment, the effective amount contains between about 0.1-5 mg/kg patient weight, as applicable. The effective dose will depend at least in part on the route of administration. The agents may be administered orally, sublingually, in an aerosol spray; by injection, e.g. i.m., s.c., i.p., i.v., etc.

In one embodiment, the cardiac glycoside(s) is delivered to the mucosal surfaces of the mouth, which could include the sublingual area, the buccal area, other areas of the mouth, or the greater oropharynx. The mouth has multiple mucosal surfaces that are rich in blood vessels and are known to rapidly uptake pharmaceutical agents. For example, the sublingual area is particularly rich in its blood supply and ability to take up pharmaceutical agents. Sublingual administration has the advantages that food ingestion and/or gastric acid do not effect the bioavailability or activity of pharmaceutical agents and that these agents appear quickly in the circulation. These advantages confer a faster onset of action with a lower dose when compared to oral administration where pharmaceutical agents must pass through some or all of the digestive tract in order for absorption to occur. The drug nitroglycerine is rapidly absorbed sublingually to quickly enter the blood stream where it affects cardiac cells. In another example, the hay-fever medicine Gravax is administered in sublingual doses comprised in fast-dissolving tablets. In another example, sublingual administration of 20 mg of sildenafil is safe and effective in the treatment of erectile dysfunction.

In one embodiment, cardiac glycosides are delivered trans-dermal by a patch. Absorption of pharmaceutical agents by the skin is an effective and non-invasive route of administration that bypasses degradation or neutralization in the gastrointestinal tract or in other routes. The number of FDA-approved polymers for use as transdermal delivery agents is increasing rapidly. In one example, a transdermal delivery patch effectively delivers scopolamine to the bloodstream through the skin behind the ear. In a second example, nicotine replacement therapy is a common over-the-counter transdermal delivery patch to treat smokers with nicotine addiction to aid in smoking cessation.

In one embodiment, cardiac glycosides are delivered by a topical drug delivery system consisting of a topical gel, foam, spray, cream, medicated powder or solution. Topical drug delivery is advantageous in that topic application is fast, easy and convenient to apply and confers improved patient compliance. The use of topical drug delivery systems for transdermal absorption of pharmacologic agents is increasingly more common in situations where pharmacologic agents are susceptible to degradation or neutralization that occur in other routes of administration. The use of topical drug delivery systems is particularly relevant in skin-associated diseases such as psoriasis, dermatitis, eczema, acne and skin infections. In one example, topical steroids such as corticosteroid hormones produced by the adrenal glands are the most frequently used treatment for psoriasis. Topical administration of corticosteroids has tremendous benefit in reducing inflammation and significantly reduces the side effects that are seen with orally administered corticosteroids. In a second example, cases of mild to moderate acne are treated by topical retinoid administration.

The periodicity of administrating effective doses of cardiac glycosides can be on a daily, weekly or on a periodic basis. In one embodiment, cardiac glycosides are administered through the sublingual, intravenous, intraparitoneal, subcutaneous, intramuscular, intrathecal, oral, rectal, nasal, ophthalmic and transdermal routes once a week and provides therapeutic effects. In a second embodiment, cardiac glycosides are administered through the aforementioned routes once every three days to provide therapeutic effects. In another embodiment, cardiac glycosides are administered once every other week to provide therapeutic benefit. In another embodiment, cardiac glycosides are administered once a month to provide therapeutic benefit.

The compositions are administered in a pharmaceutically acceptable excipient. The term “pharmaceutically acceptable” refers to an excipient acceptable for use in the pharmaceutical and veterinary arts, which is not toxic or otherwise inacceptable. The concentration of cardiac glycoside in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

### Optimizing Dosing and Selection of Agents

Candidate agents of interest are biologically active cardiac glycoside agents, which may be selected from the known compounds in this class, or derivatives and analogs thereof. The agent may also include testing of route, dose and regimen for various formulations comprising cardiac glycosides, where formulations may vary in excipients such as pH buffers, stabilizing agents, and the like.

The candidate agent or formulation is screened for activity optimization by adding the agent to a population of cells comprising B cells, and including regulatory B cells or progenitors thereof, e.g. PBMC, and the like. Typically control and reference agents and cell populations will be included. The change in B cell regulatory activity in response to the agent is measured, desirably normalized, and the resulting activity may then be evaluated by comparison to reference cells and/or agents. Those agents, formulations and regimens that provide for increased B cell regulatory activity are desirable candidates for use in the methods of the invention, where analysis may include detection of cytokine secretion by any convenient method, or biological activity, e.g. determining an effect on T cells regulated by the B cells. In some embodiments the cells are provided in vivo. In other embodiments an in vitro culture system is used.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agents, formulation, doses, etc. to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the cytokines indicative of regulatory B cells. For measuring the amount of a molecule that is present, a convenient method is to label a molecule with a detectable moiety, which may be fluorescent, luminescent, radioactive, enzymatically active, etc., particularly a molecule specific for binding to the parameter with high affinity Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody- or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

This invention will be better understood by reference to the Examples which follow, but those skilled in the art will readily appreciate that the information detailed is only illustrative of the invention as described more fully in the claims which follow thereafter.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference.

### EXPERIMENTAL

### Example 1

### Cardiac Glycosides Treat Rheumatoid Arthritis (RA)

It is demonstrated herein that digitoxin, a plant-derived member of the cardiac glycosides, and ouabain and proscillaridin A, two endogenous cardiac glycosides in humans, potently prevent and treat collagen-induced arthritis (CIA), a murine model for rheumatoid arthritis.

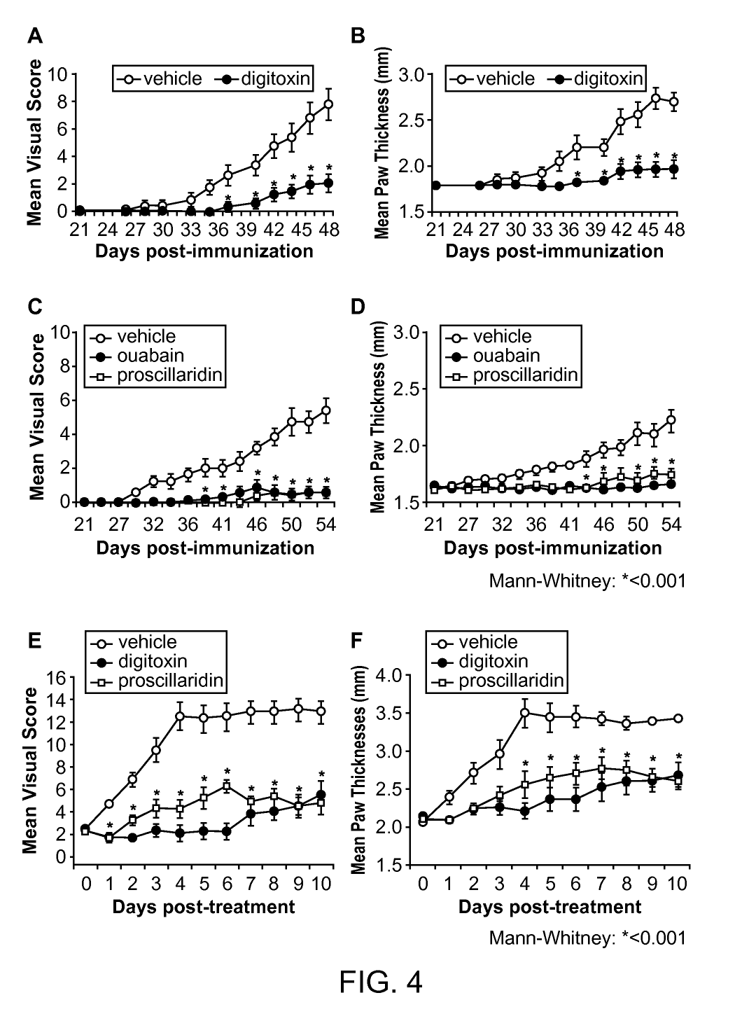

It was initially determined that the cardiac glycosides were able to prevent the onset of CIA in DBA1/J mice. CIA was prevented in cardiac glycoside recipients as compared to vehicle recipients (FIG. 4 A-D). In prevention studies (FIG. 4 A-D), DBA1/J 6-7 week old male mice received daily doses of 0.3 mg/kg digitoxin (FIGS. 4 A and B), 0.03 mg/kg ouabain (FIGS. 4 C and D) or 0.25 mg/kg proscillaridin (FIGS. 4 C and D) by intra-peritoneal injection the starting the day prior to intradermal immunization with 100 ug/mouse bovine collagen II-(Chondrex) in complete Freund's adjuvant. 21 days later, mice were boosted by subcutaneous injection at the base of the tail with 100 ug/mouse bovine collagen II in incomplete Freund's adjuvant. Mice are visually scored (FIGS. 4 A and C) by the following criteria: 0=no swelling or erythema, 1=mild swelling and erythema or digit inflammation, 2=moderate swelling and confined distal erythema, 3=pronounced swelling and erythema extending toward the ankle, 4=severe swelling and erythema with joint rigidity of the ankle, foot, and digits. Each limb is graded with a score of 0-4, where the maximum score for a mouse is 16. Paw thickness was determined by caliper measurements (FIGS. 4 B and D).

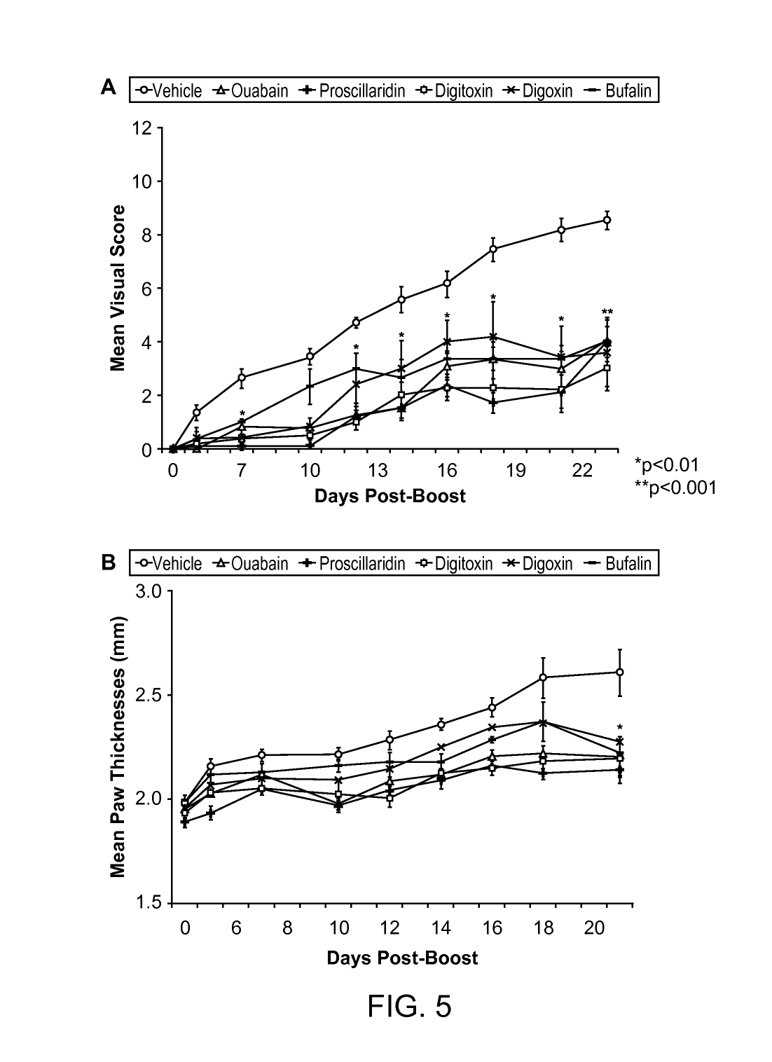

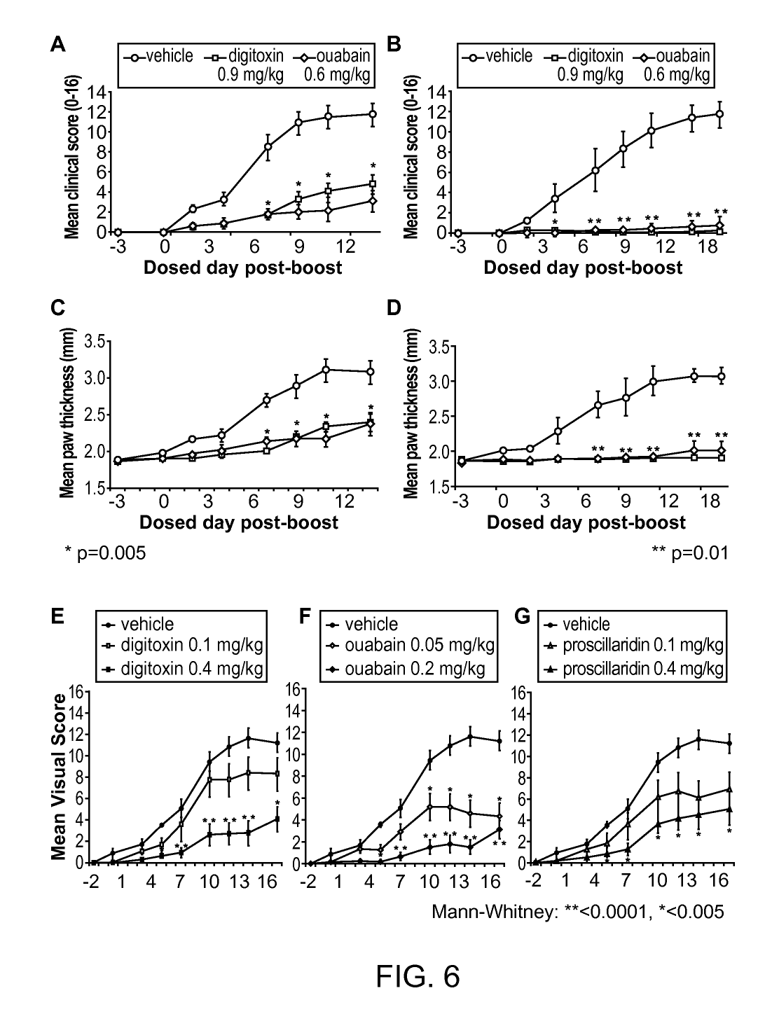

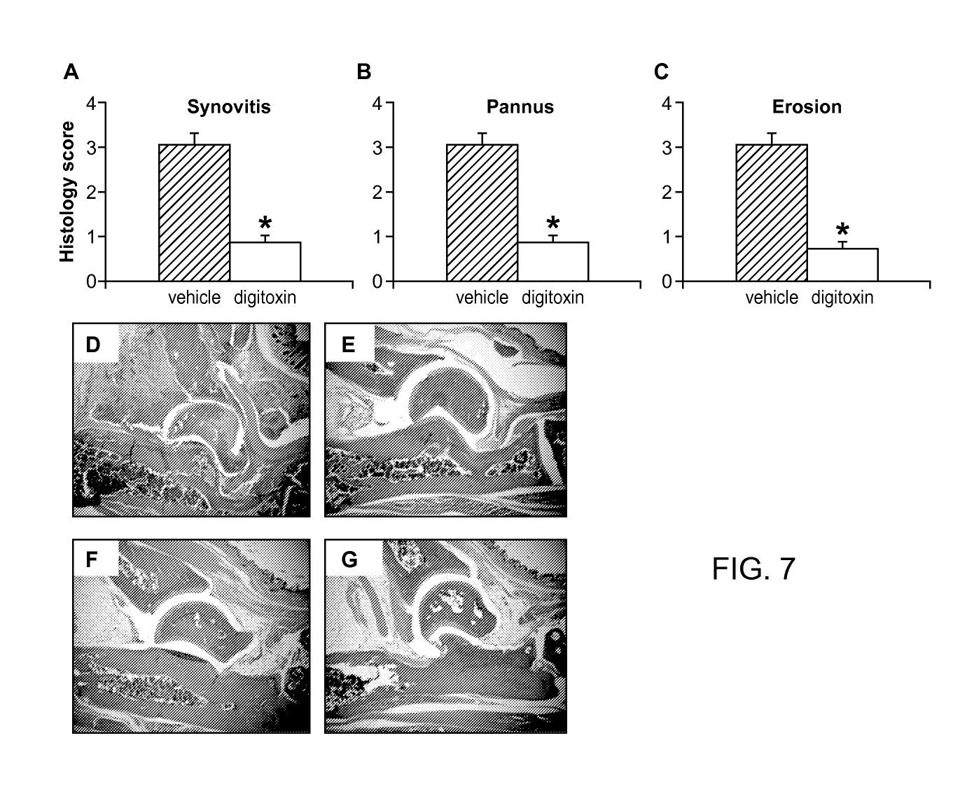

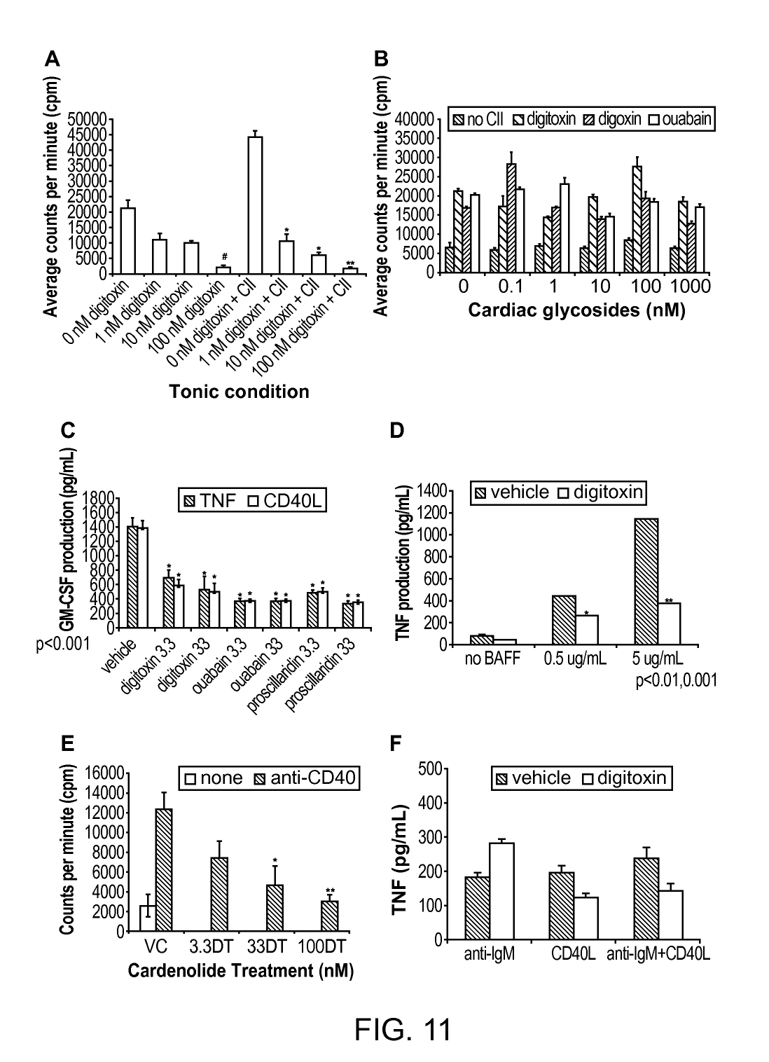

In treatment studies, mice were randomly enrolled in treatment groups and treatment commenced when the average clinical score was at or above 2. In treatment studies, mice were enrolled randomly into groups when they reached an average clinical score of 2. Daily dosing commenced upon enrollment at the abovementioned concentrations. Mice receiving daily cardiac glycoside dosing had markedly reduced clinical scores and joint inflammation compared to vehicle control mice (FIGS. 4 E and F). The ability of multiple members from cardenolide (digitoxin, ouabain, digoxin) and bufadienolide (proscillaridin, bufalin) classes was assessed and multiple were found to significantly reduce clinical manifestations of collagen arthritis in a pro-treatment format; whereby mice were randomized into groups and initially dosed with cardiac glycoside or vehicle one day prior to boost (FIGS. 5 A and B). Administration routes and dosing periodicity were also investigated and found that mice dosed every-three-days with 0.9 mg/kg digitoxin or 0.6 mg/kg ouabain by i.p. injection had significantly less disease and inflammation than the vehicle mice (FIGS. 6 A and C). Additionally, mice were administered cardiac glycoside every-three-days by sublingual administration and showed that 0.9 mg/kg digitoxin and 0.6 mg/kg ouabain significantly inhibited the onset of collagen arthritis (FIGS. 6 B and D). Dose titration studies demonstrate a dose-effect in vivo (FIG. 6 E-G). Double-blinded histopathology scoring was conducted of cross-sectioned joints taken from cardiac glycoside and vehicle recipients. Cardiac glycoside-treated mice had a significantly lower occurrence of synovitis, pannus, and bone erosion than vehicle recipients (FIG. 7 A-C). Hematoxylin and eosin stains of cross-sectioned ankle joints also demonstrate the decreased amounts of immune cell infiltrate into the joints of digitoxin (FIG. 7 E), ouabain (FIG. 7 F) and proscillaridin (FIG. 7G) but not vehicle (FIG. 7 D) treated mice. Further characterization of cardiac glycoside-treated mice revealed significantly decreased serum immunoglobulin levels of IgM and IgG1 isotypes, but not IgG2a (FIGS. 10 A and B). The results obtained from these animal studies indicate that cardiac glycosides are effective at reducing the clinical manifestations of disease and reducing joint pathology in mice with collagen arthritis.

### Methods:

Clinical effects of cardiac glycosides on CIA. CIA was induced in 6-7 week old male DBA/1 mice by intra-dermal tail immunization with bovine CII (Chondrex) at 100 mg/mouse, emulsified in CFA containing 250 mg/mouse heat-killed M. tuberculosis H37Ra (BD). Twenty-one days after immunization, mice were boosted by subcutaneous injection of 100 mg/mouse bovine CII emulsified in IFA. For prevention studies, mice received daily glycoside treatment one day prior to initial immunization and throughout the duration of the experiment. In treatment studies, daily glycoside treatment was administered to mice with an average disease score of 2. Mice were visually scored by the following criteria: 0=no swelling or erythema, 1=mild swelling and erythema or digit inflammation, 2=moderate swelling and confined distal erythema, 3=pronounced swelling and erythema extending toward the ankle, 4=severe swelling and erythema with joint rigidity of the ankle, foot, and digits. Each limb was graded with a score of 0-4, where the maximum score for a mouse is 16.

TABLE
Examples of cardiac glycosides.

| Glycoside | Source | Murine dosing (ip) |
|---|---|---|
| digitoxin | Fluka #37030 | 0.3 mg/kg |
| digoxin | Sigma #D6003 | 0.3 mg/kg |
| dihydroouabain | Sigma #D0670 | 0.2 mg/kg |
| ouabain | Fluka #75640 | 0.06 mg/kg |
| proscillaridin | Sigma #P2428 | 0.25 mg/kg |
| Strophanthidin | Sigma # S6626 | Experimentally determined |
| Isostrophanthidin | Synthesized | Experimentally determined |
| Saponified | Synthesized | Experimentally determined |
| isostrophanthidin | | |
| Strophanthidin | Sigma # S6626 | Experimentally determined |
| Isostrophanthidin | Synthesized | Experimentally determined |
| Strophanthidinic acid | Synthesized | Experimentally determined |
| Pseudostrophanthidin | Synthesized | Experimentally determined |
| Isostrophanthidin | Synthesized | Experimentally determined |
| Allostrophanthidin | Synthesized | Experimentally determined |
| Cymarin | Sigma # 30030 | Experimentally determined |
| Allocymarin | Synthesized | Experimentally determined |

The effects of the different cardiac glycoside treatments on CIA pathology was analyzed by histological scoring. Inflammatory cell infiltrate, synovial hyperplasia, pannus formation and articular cartilage and bone deterioration were evaluated in sectioned paws of treated mice at specific time-points throughout the disease course. Histopathology scores of inflammation, pannus formation and bone and cartilage erosions were conducted by a blinded investigator on toludine blue-stained cross sections of fixed leg joints from representative mice in vehicle and glycoside groups. Pathology scoring for each of the three categories are on a scale of 0-4 with grade 0=normal, 1=mild inflammation, hyperplasia of the synovial lining, cartilage destruction without bone erosion, grades 2 to 4=increasing degrees of inflammatory cell infiltrate, synovial hyperplasia, pannus formation and cartilage and bone destruction. Error bars represent SEM and statistics were performed using the Mann-Whitney test. At the endpoint, mice are bled and serum is collected from representative mice and stored at −80 C until use. To determine anti-collagen II antibody levels and to characterize isotype prevalence, bovine collagen II (Chondrex) was coated O/N at 4 C on an ELISA plate, washed and incubated with 100 uL of 1:10 dilution of each serum sample (in triplicate) for 2 h at RT. Wells were washed and incubated with anti-mouse IgM u-chain specific (Jackson), anti-mouse IgG1, or anti-mouse IgG2a (Southern Biotech) for 1 h at RT. The ELISA was developed and read using BD ELISA development kit. Statistical analysis was performed using the two-tailed ANOVA and asterisks represent P-values less than 0.01.

### Example 2

### Cardiac Glycosides Inhibit the Activity of TNFR Family Members

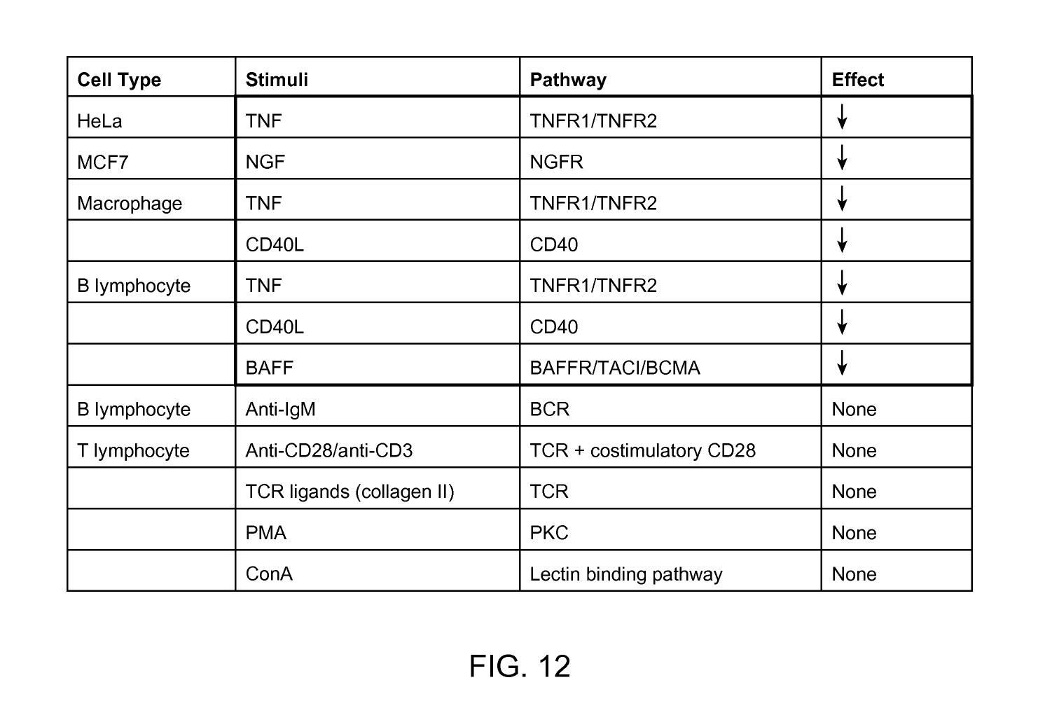

Therapeutic concentrations determined in the CIA experiments (example 1) are the same concentrations of cardiac glycosides that are found to inhibit effector functions of innate immune cells isolated from chronically-inflamed CIA mice (FIG. 11). Cardiac glycosides inhibit NFκB activity through the abrogation of downstream signaling molecules from multiple members of the TNF receptor (TNFR) family: CD40, BAFFR/BCMA/TACI and TNFR1 (FIGS. 11 and 12).

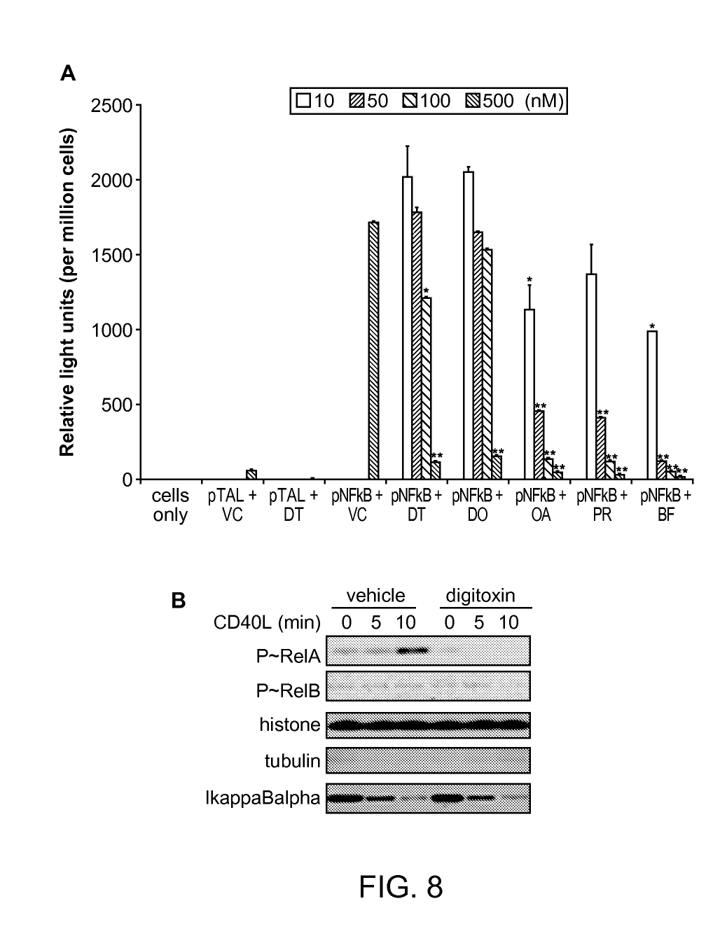

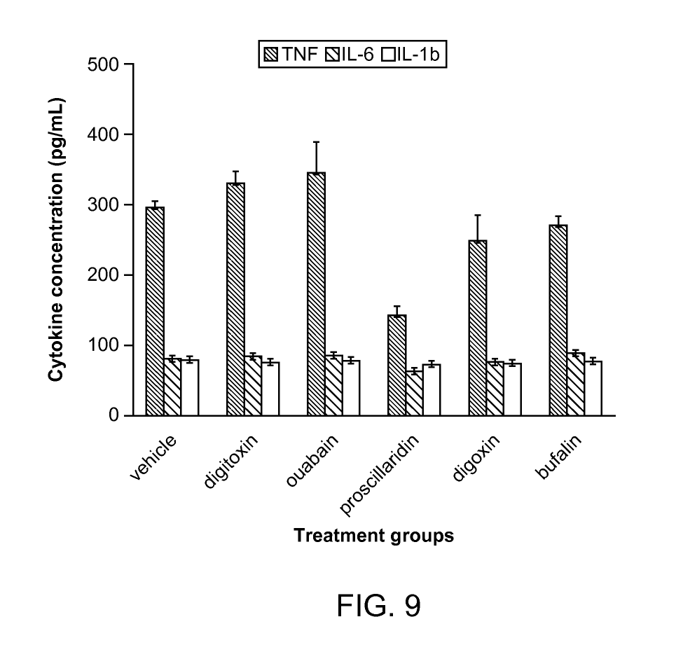

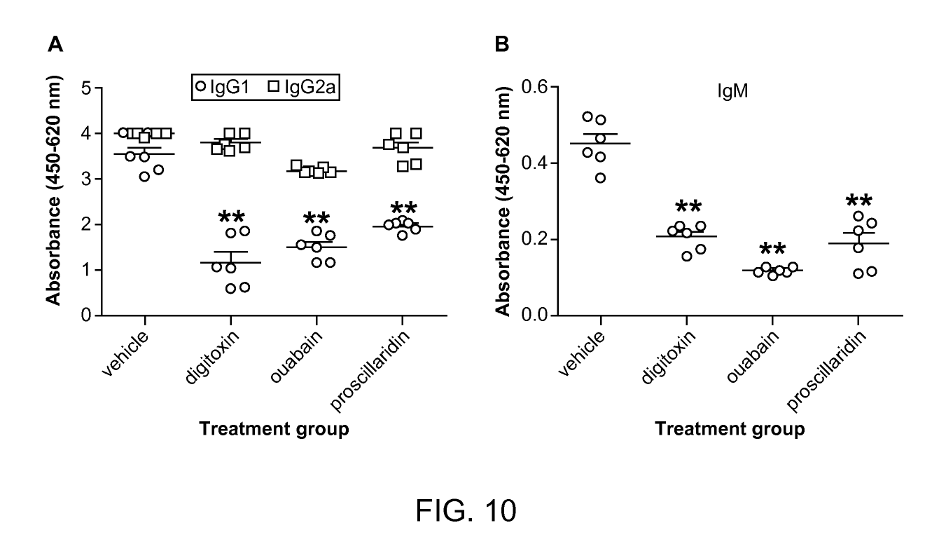

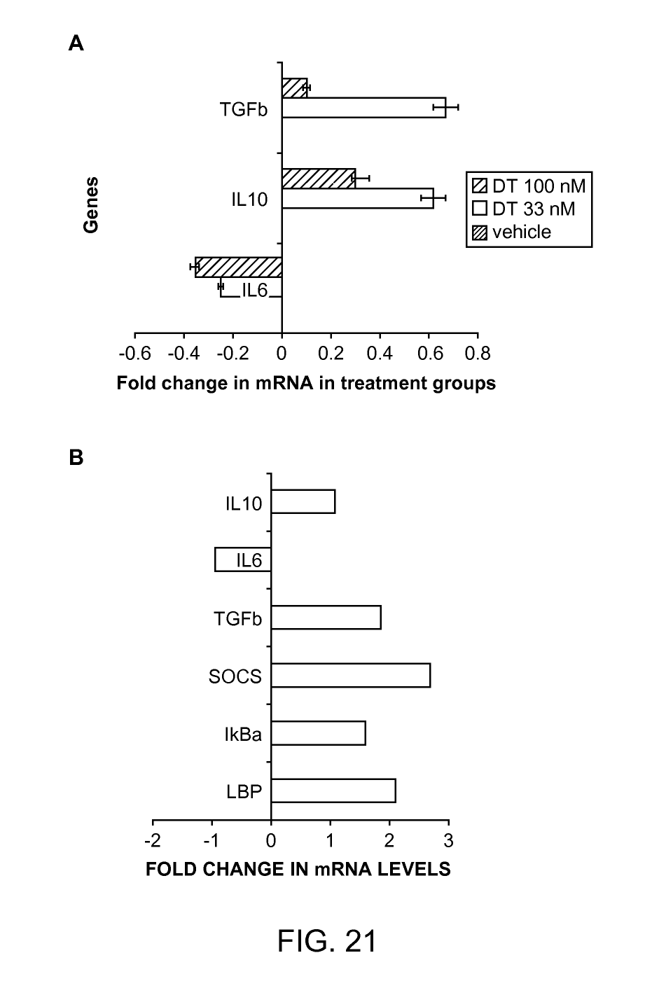

Cardiac glycosides effectively inhibit TNF-mediated NFκB activity in HeLa cells transiently transfected with an NFκB-responsive luciferase reporter (Clontech). HeLa cells were co-transfected with the reporter pNFκB-luc and control vector pTAL (Renilla) for 18 h prior to incubation with 0-500 nM cardiac glycoside for 6 h. Cells are stimulated with 50 ng/mL TNF. Cardiac glycosides were assessed for the ability to inhibit NFκB activity and for the ability to demonstrate a dose-dependent inhibition of TNF/TNFR-mediated NFκB activity using the NFκB-luciferase reporter system (FIG. 8 A). Anti-phospho-p65 Western immunoblots were performed on nuclear fractions of B cells taken from CIA mice that were stimulated with 50 ng/mL CD40L to validate and expand on the luciferase assay results. FIG. 8 B). Cardiac glycoside treatment can also induce STAT3-dependent IκBα gene expression that can have effects on NFκB activity (FIG. 21).

Cardiac glycosides inhibit CIA splenocyte activity in a dose-dependent manner (FIG. 11 A). Activity was monitored by ³H-thymidine incorporation as a read-out for proliferation. Cardiac glycosides inhibited CIA splenocyte activity in the presence of activating antigen, collagen II (CII) (FIG. 11A). Cell type(s) in the splenocyte population responsive to therapeutic concentrations of cardiac glycoside were determined by identifying the population of cells that responded to a dose-dependent decrease in activity in the presence of stimulant, defined as ligand(s) binding to cognate receptor(s). Activity was analyzed by: ³H-thymidine or BrdU incorporation to monitor proliferation or by ELISA to monitor cytokine production. Splenic T lymphocytes were isolated by negative selection (MACS, Miltinyi Biotec) and are stimulated with PMA, ConA or through the TCR with anti-CD3/anti-CD28 Dynabeads (Invitrogen) and collagen II (all non-TNFR receptors/pathways). At therapeutic concentrations, cardiac glycosides did not affect signaling through PMA-, ConA- and TCR-stimulated pathways (FIG. 11 B; FIG. 12). Peritoneal and bone marrow-derived macrophages were isolated from CIA mice in the presence of M-CSF growth factor and were stimulated with TNF and CD40 ligand (CD40L). At therapeutic concentrations, cardiac glycosides inhibited macrophage activation through the TNFR family members TNFR and CD40 in a dose-dependent manner (FIG. 11 C). Splenic B lymphocytes were isolated by negative selection (MACS, Miltinyi Biotec) and were stimulated with BAFF (BLyS), CD40L or through the BCR with anti-IgM u-chain specific antibodies. At therapeutic concentrations, cardiac glycosides inhibited B cell activity in a dose-dependent manner when stimulated with ligands for the TNFR family members (FIG. 11 D-F). In contrast, stimulation of the B cell receptor (BCR), a non-TNFR family receptor, was not inhibited by therapeutic concentrations of cardiac glycosides (FIG. 11 F). Downstream signaling from multiple TNFR family members was inhibited by therapeutic concentrations of cardiac glycosides. In contrast, therapeutic concentrations of cardiac glycosides did not alter stimulation through TCR, BCR, ConA and PMA, non-TNFR family receptors/pathways.

Cardiac glycoside cytotoxicity studies were performed with primary purified T lymphocytes, B lymphocytes, macrophages and splenocytes. Cytotoxicity studies include trypan blue staining, Annexin V-staining, caspase-3 cleavage ELISAs and adenylate kinase release assays. 0-100 nM cardiac glycosides were not cytotoxic or cytolytic to primary cells suggesting a non-cytotoxic mechanism for inhibition (data not shown).

Therapeutic concentrations of cardiac glycosides inhibit immune cell activation at supra-physiologic levels of TNFR ligands. Cardiac glycosides can be administered to patients with high tissue, serum or plasma levels of TNF ligand superfamily members or to patients with abnormally active TNFRs to inhibit activity of downstream immune cell-specific signaling pathways in a dose-dependent manner for the abrogation of inflammatory responses.

The findings suggest that cardiac glycoside treatment can be used to inhibit signaling from TNFR family members, such as (but not limited to): TNFR1, TNFR2, FAS, CD30, CD27, CD40, CD137, CD120a, CD120b, lymphotoxin-beta receptor, CD134, TNFRSF6B, TNFRSF10A, INFRSF10B, TNFRSF10C, TNFRSF10D, RANK, osteoprotegerin, TNFRSF12A, TNFRSF13B, TNFRSF13C, TNFRSF14, nerve growth factor receptor, TNFRSF17, TNFRSF18, TNFRSF19, TNFRSF21, TNFRSF25 and ectodysplasin A2 receptor.

### Example 3

### Cardiac Glycosides for the Treatment of Systemic Lupus Erythematosus (SLE)

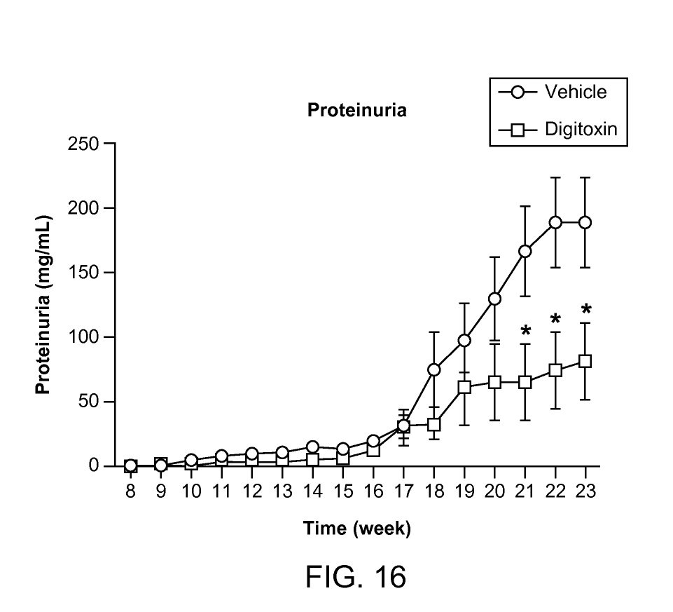

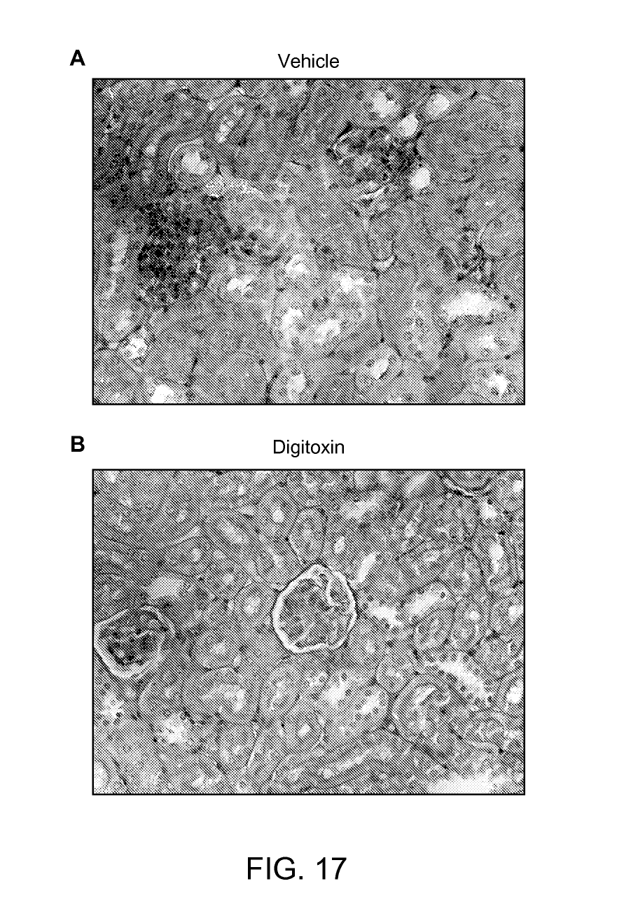

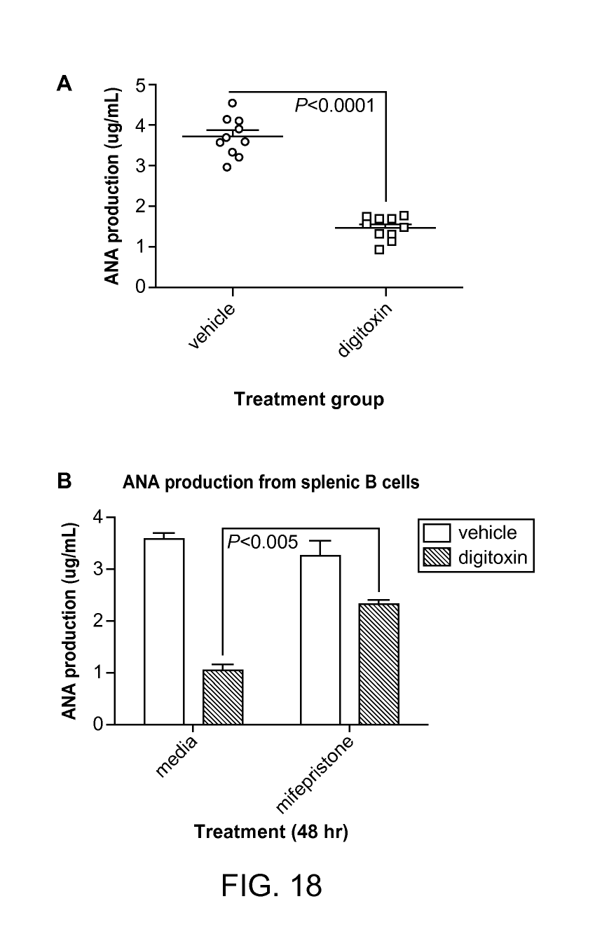

Cardiac glycosides are used for the treatment of systemic lupus erythematosus and other forms of multi-system inflammatory diseases. To demonstrate their effectiveness, the experimental model for systemic lupus erythematosus (SLE) is used. Studies are conducted to determine the ability of cardiac glycosides to treat SLE. The effectiveness of cardiac glycosides at treating SLE disease progression in MRL-Ipr female mice (Jackson Laboratories) was evaluated. Briefly, 10 mice per experimental arm were followed for the development of SLE disease. At week 8, mice were dosed with vehicle or 0.6 mg/kg digitoxin every-4-days by sublingual administration. Animals were scored on a weekly basis for proteinuria and survived until 23 weeks of age (FIG. 16). Weekly proteinuria monitoring was performed using Uristix (Bayer Corp.). At endpoint, mice were sacrificed and harvested for splenocytes, serum and kidneys. Kidneys were preserved in fixative, embedded in paraffin, sectioned longitudinally and stained for glomeruli with periodic acid-Schiff stain (Histotec) (FIGS. 17 A and B). Endpoint serum titres of double-stranded DNA (ANA) antibodies for each mouse were assessed (FIG. 18 A). Additional tests were performed on MRL-Ipr splenic vehicle B cells to determine the ability of digitoxin to inhibit ANA production in vitro (FIG. 18 B). ANA antibodies were measured using ELISA (Alpha Diagnostic International). The Mann-Whitney and student t-test was used to determine significance between vehicle and digitoxin groups in animal and endpoint studies, respectively. The results obtained from these animal studies indicate that cardiac glycosides are effective at reducing the clinical manifestations of SLE disease and reducing kidney pathology in mice with SLE.

### Example 4

### Cardiac Glycosides for the Treatment of Myasthenia Gravis (MG)

Cardiac glycosides are used for the treatment of myasthenia gravis and other antibody-mediated autoimmune diseases. To demonstrate the effectiveness of cardiac glycosides in the prevention and treatment of myasthenia gravis, the experimental autoimmune myasthenia gravis (EAMG) model is used. 200 ug of purified acetylcholine receptor (AChR) from Torpedo californica is emulsified in complete Freund's adjuvant (CFA) and is injected subcutaneously in each of four sites (50 uL per hind foot-pad and 50 uL per shoulder) achieving 200 uL/mouse, 20 ug AChR in 8-10 week old C57BL6/J male mice (Jackson Laboratories). A second immunization is performed in the same manner as the initial immunization 30 days after the first. EAMG-related muscle weakness is evaluated daily by paw-grip testing. EAMG scores are the following: grade 0, normal muscle strength and no muscle weakness before or after exercise (exercise=20 to 30 consecutive paw grips to a steel grid cage top); grade 1, normal at rest but weak after exercise, with chin on the floor and inability to raise head, hunched back and reduced mobility; grade 2, weakness at rest; grade 3, moribund, dehydrated and paralyzed (quadriplegic). Mice are bled at the endpoint and anti-AChR antibody titres are determined by ELISA. The Mann-Whitney test is used to determine if mice treated with cardiac glycosides exhibit less severe EAMG as compared to vehicle recipient mice, and data are displayed as mean muscle weakness per group and mean anti-AChR antibodies per group.

### Example 5

### Identification of Individuals with Low Levels of Endogenous Cardiac Glycosides and the Administration of Cardiac Glycoside Supplementation

Endogenous cardiac glycoside reactivity (ECGR) can be detected by anti-cardiac glycoside ELISA on human samples (FIG. 13 A), and absolute concentrations of each endogenous cardiac glycoside can be determined by liquid chromatography-mass spectrometry, ion electrospray mass spectrometry (FIG. 13 B), fast atom bombardment mass spectrometry and proton nuclear magnetic spectroscopy methods. Patient fluids (plasma, urine, serum) and tissues are analyzed for ECGR and compared to healthy samples. Cardiac glycosides are used to supplement patients with undetectable or low levels of endogenous cardiac glycosides, such as in Addison's disease, X-linked adrenoleukodystrophy, ankylosing spondylitis, rheumatoid arthritis, or other diseases in which the hypothalamus, pituitary or adrenal glands have lost function or are not producing optimal amounts of the natural cardiac glycosides. The elevated ECGR levels in irritable bowel disease patient sera teaches against the use of cardiac glycoside as treatment for all autoimmune diseases or all diseases associated with inflammation. ECGR assessment is important to determine cardiac glycoside levels prior to administration. Treatment of autoimmune diseases, and specific patients with autoimmune disease, who manifest low cardiac glycoside levels relative to the population as a whole or to a normal reference range may provide therapeutic benefits, as demonstrated in mouse models for multiple autoimmune diseases.

### Example 6

### Cardiac Glycosides for the Modulation of STATs and Other Transcription Factors Involved in Inflammatory Signaling

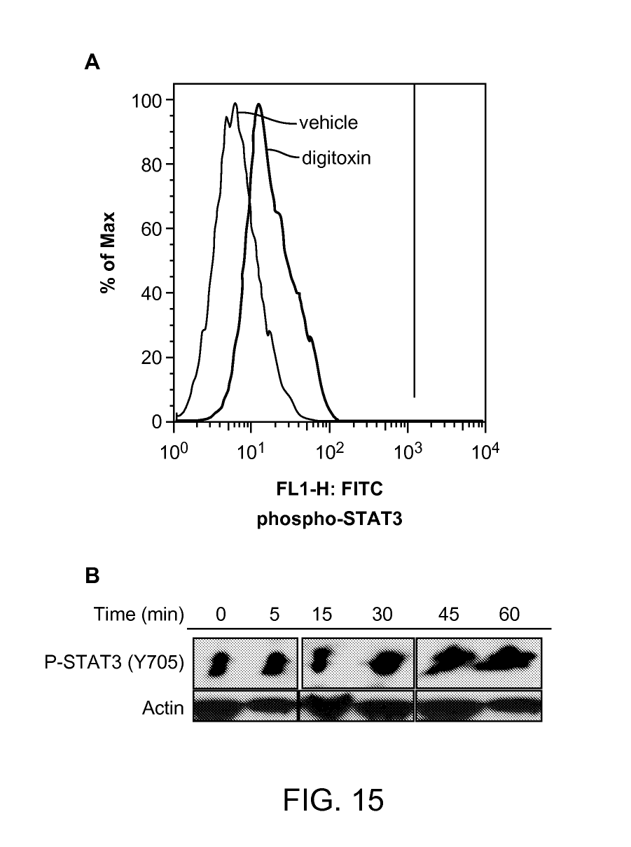

We performed studies in which we demonstrate that cardiac glycosides modulate the activity of signal transducer and activator of transcription (STAT). Cardiac glycosides were administered at endogenous levels (sub- and nano Molar) or therapeutic levels (nano Molar) to modulate transcription factor activity and gene expression. Alteration of STAT1 activity has been observed in Alzheimer's disease and susceptibility to lethal intracellular pathogenic diseases (Kitamura et al, 1997; Chapgier et al, 2009). Aberrant STAT2 levels have been linked to inflammatory disease such as Crohn's disease (CD), ulcerative colitis (UC) and inflammatory bowel disease (IBD), and cancers such as colorectal and skin cancers (Gamero et al, 2010). STAT3 signaling is a critical component of Th17-dependent autoimmune processes. Genome-wide association studies (GWAS) have revealed the role of the STAT3 gene in IBD, CD, ulcerative colitis (UC) and multiple sclerosis (MS) susceptibility, although confirmation in clinical sub-phenotypes is warranted. Mice with targeted deletion of Stat3 in T cells are resistant to experimental autoimmune encephalomyelitis, which is a multiple sclerosis (MS) model. Increased phosphorylated STAT3 was reported in T cells of patients evolving from clinically isolated syndrome to defined MS and in relapsing patients. These evidences led us to analyze the role of STAT3 in CD, UC and multiple sclerosis (MS) risk. A proinflammatory IL6-STAT3 biologic network is upregulated in active pediatric IBD patients at diagnosis and during therapy. Specific targeting of this network is effective in reducing mucosal inflammation. Cardiac glycosides are administered to individuals or cells (FIG. 15) to modulate STAT phosphorylation and the immune response. Cardiac glycoside treatment can also induce STAT3-dependent IκBα expression to down-regulate NFκB activity (FIG. 21). STAT phosphorylation can be assessed by phospho-flow cytometric analysis, Western immunoblot and ELISA of circulating PBMCs or of biopsied tissues.

### Example 7

### Cardiac Glycosides for the Induction of Cytokine Gene Expression and Cytokine Protein Production

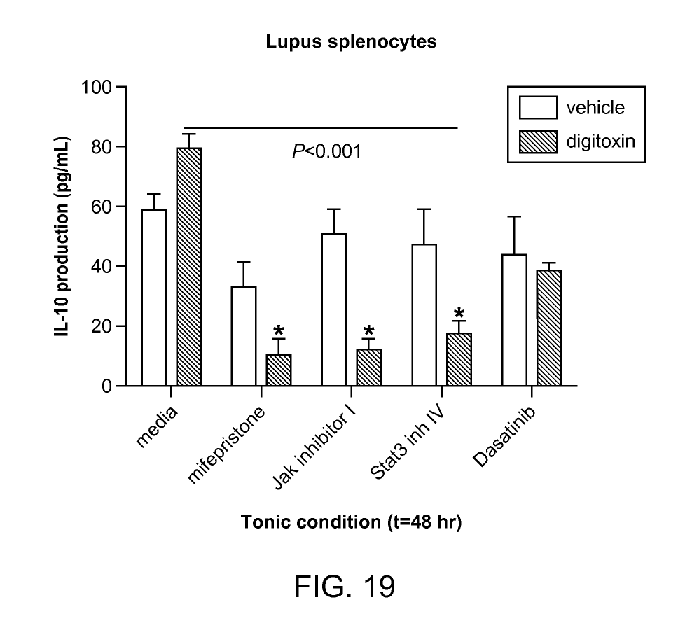

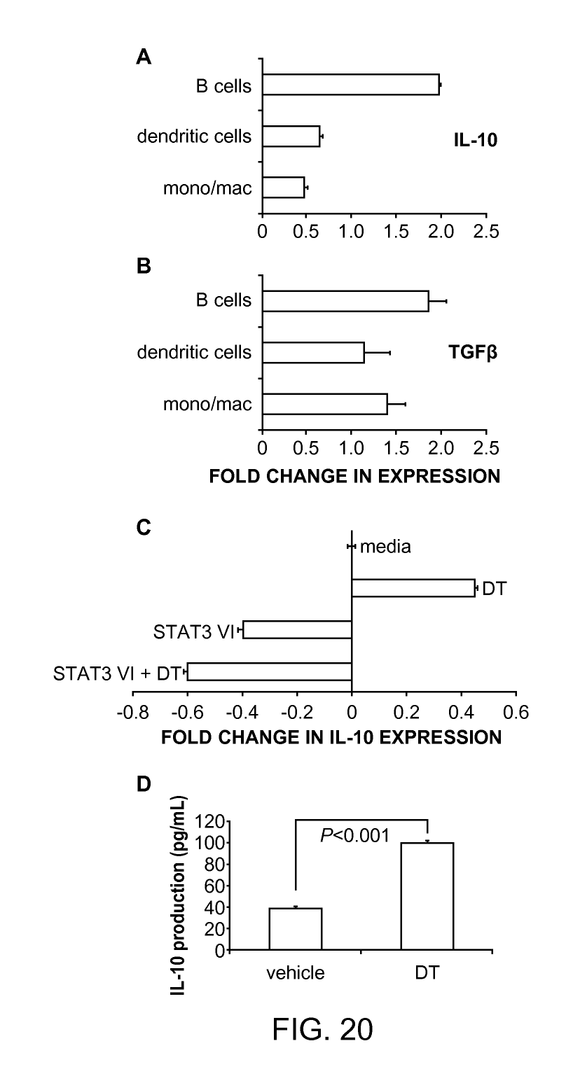

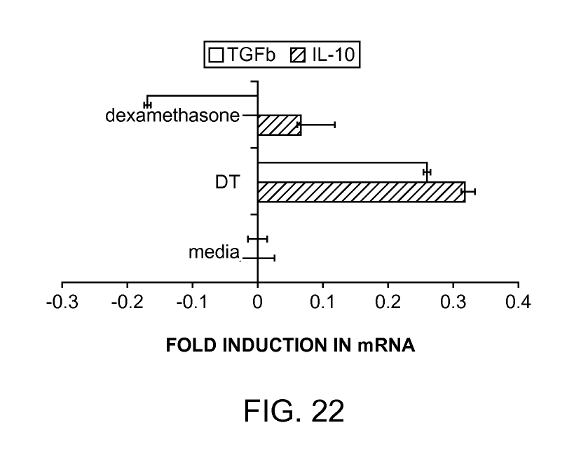

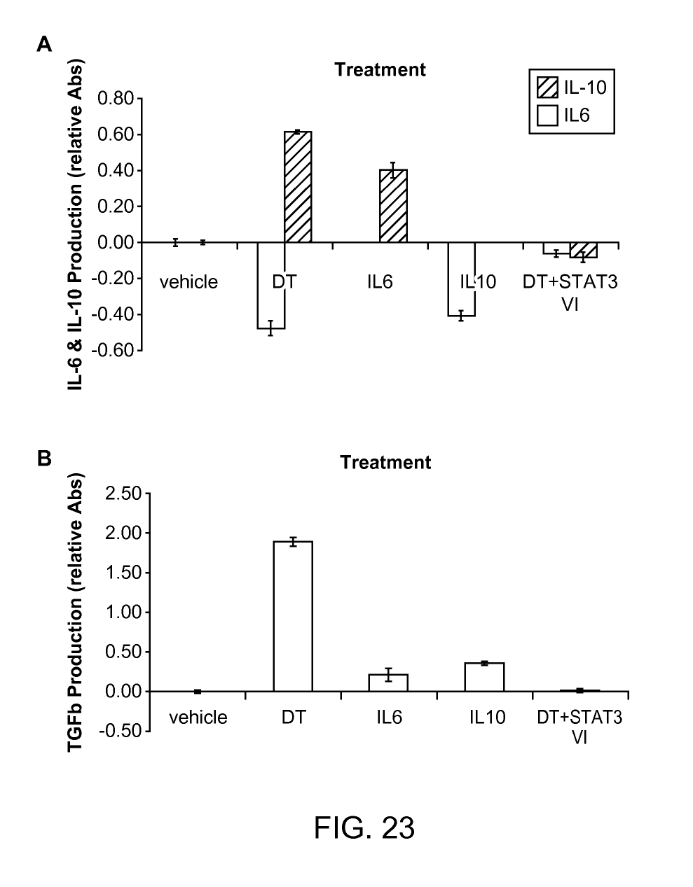

It has been reported that cardiac glycosides inhibit cytokine secretion (U.S. Pat. No. 5,545,623 and U.S. patent application Ser. No. 12/229,399). We performed gene expression studies using ELISA and RT-PCR on B cells, dendritic cells and monocyte/macrophages purified from mice with CIA and lupus in the absence or presence of digitoxin and ouabain. (FIGS. 19, 20, 21, 22, and 23). Cardiac glycoside was demonstrated to induce IL-10 and TGF cytokine gene expression in these cells and superior IL-10 and TGFβ cytokine gene expression was observed in B cells (FIGS. 20 A and B). The kinetics of IL-10 and TGFβ cytokine gene expression was rapid, with cardiac glycoside-dependent induction of gene expression occurring as early as 30 minutes following its administration (FIGS. 20 A and B and 21 A). Additional genes, SOCs3, IκBα, LPS binding protein (LBP) and others were found to be up-regulated within 45 minutes after cardiac glycoside administration (FIG. 21 B). Cardiac glycoside-induced IL-10 gene expression was determined to be dependent on STAT3 activity (FIG. 19 and FIG. 20 A and FIGS. 23 A and B). Importantly, other steroids, such as dexamethasone were found to induce IL-10 cytokine but not TGFβ cytokine expression. Cardiac glycosides were capable of inducing the expression of both IL-10 and TGFβ cytokine expression (FIG. 22). Thus, cardiac glycoside treatment induces expression of the cytokines IL-10 and TGFβ, which have anti-inflammatory properties.

### Example 8

### Cardiac Glycosides Induce IL-10 Producing B Cells that Regulate T Cell Differentiation

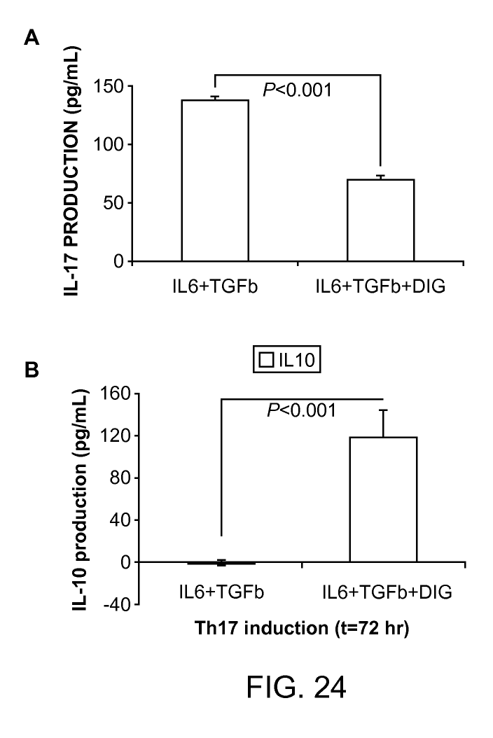

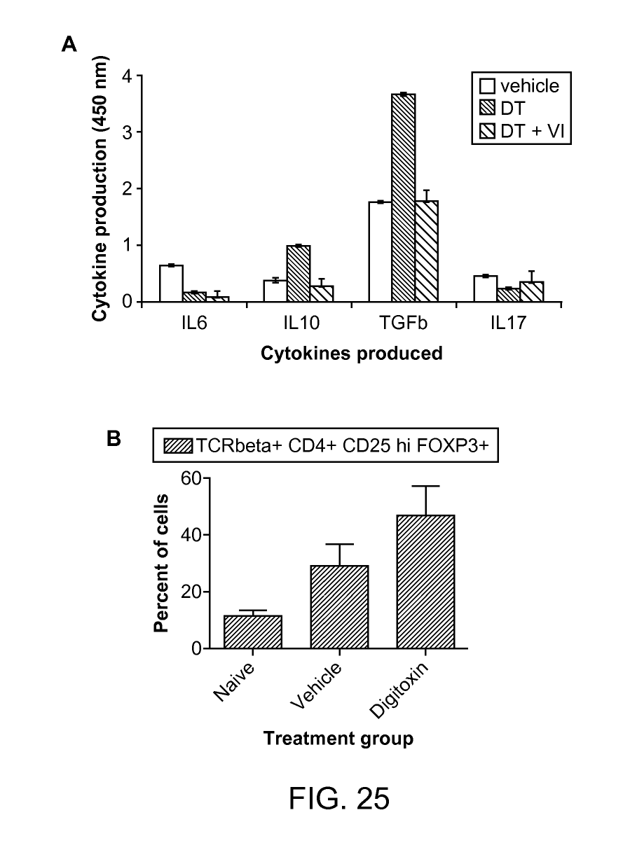

It is known that B cells can regulate T cell differentiation and in many states of autoimmune disease, an imbalance of Th1, Th2, and Th17 T cell populations exist. Here we present data demonstrating the administration of cardiac glycosides to B cells can modulate T cell fate. We performed studies to determine the regulatory effector function of B cells pre-treated with cardiac glycoside on T cell differentiation. B cells blocked naïve CD4⁺ T cell differentiation into Th17 cells when cardiac glycoside was administered to B cells 30 minutes prior to co-culture with T cells (FIG. 24 A). Additionally, the pre-treatment of B cells with cardiac glycoside inhibited Th17 differentiation in a STAT3-dependent manner (FIG. 25A). Furthermore, increased differentiation of T cells into regulatory T cells was demonstrated in cardiac glycoside treated animals (FIG. 25 B). Cardiac glycosides can be administered to patients to induce IL-10 expressing B cells and to modulate T cell differentiation and increase numbers of Tregs which reduce autoimmune disease progression.

1. A method for treating an inflammatory disease in an individual, the method comprising:
administering to the individual a dose of a cardiac glycoside by a route and dosing regimen that is effective in increasing expression of at least one anti-inflammatory cytokine by endogenous immune cells.

2. The method of claim 1, wherein the anti-inflammatory cytokine is increased in expression by at least two-fold relative to an untreated control.

3. The method of claim 2, wherein the anti-inflammatory cytokine is IL-10.

4. The method of claim 2, wherein the anti-inflammatory cytokine is TGFβ.

5. The method of claim 2, wherein the endogenous immune cell is a regulatory B cell.

6. The method of claim 1, wherein the inflammatory disease is rheumatoid arthritis.

7. The method of claim 6, wherein the individual has been diagnosed as having a pre-clinical stage of disease.

8. The method of claim 1, wherein the inflammatory disease is systemic lupus erythematosus.

9. The method of claim 1, wherein the inflammatory disease is myasthenia gravis.

10. The method of claim 1, wherein the cardiac glycoside is a bufadienolide.

11. The method of claim 10, wherein the bufadienolide is bufalin, telocinobufagenin, marinobufagin, marinobufagenin or proscillaridin.

12. The method of claim 1, wherein the cardenoloide is ouabain, digitoxigenin, digoxigenin, gitaligenin, ouabagenin, G-strophanthidin, digoxin, gitoxigenin, oleandrin, oleandrigenin, strodival, strophalen, strophantinum G, strophoperm, strophanthidin, strophanthin K, strophosan, strophosid, uabaina, uabanin, proscillan, proslladin, purgoxin, sandoscill, scillacrist, caradrin, caradrine, carmazon, deslanoside, dihydroouabain, scillarenin, scillaridin A, and cymarin.

13. The method of claim 1, wherein the cardiac glycoside is administered orally.

14. The method of claim 1, wherein the cardiac glycoside is administered sublingually.

15. The method of claim 14, wherein the sublingual administration is daily.

16. The method of claim 14, wherein the sublingual administration is every three days.

17. The method of claim 14, wherein a sublingual therapeutically effective dose is in the range of 0.001 to 5 mg/kg per dose.

18. The method of claim 14, wherein a sublingual therapeutically effective dose is in the range of 0.01 to 0.5 mg/kg per dose.
